(19) United States
(12) Patent Application Publication
(10) Pub. No.: US 2023/0329542 A1
(43) Pub. Date: Oct. 19, 2023

(54) PERCUTANEOUS RENAL ACCESS SYSTEM

(71) Applicant: RetroPerc, Inc., Passaic, NJ (US)

(72) Inventor: Jason B. Wynberg, Passaic, NJ (US)

(73) Assignee: RetroPerc, Inc., Passaic, NJ (US)

(21) Appl. No.: 18/207,713

(22) Filed: Jun. 9, 2023

Related U.S. Application Data
(63) Continuation-in-part of application No. 17/860,176, filed on Jul. 8, 2022.
(60) Provisional application No. 63/391,785, filed on Jul. 24, 2022.
(60) Provisional application No. 63/391,771, filed on Jul. 24, 2022.
(60) Provisional application No. 63/351,463, filed on Jun. 13, 2022.
(60) Provisional application No. 63/315,578, filed on Mar. 2, 2022.
(60) Provisional application No. 63/254,101, filed on Oct. 9, 2021.
(60) Provisional application No. 63/254,128, filed on Oct. 10, 2021.
(60) Provisional application No. 63/254,503, filed on Oct. 11, 2021.

(51) Int. Cl. A61B 1/307; A61B 1/018

(52) U.S. Cl. CPC A61B 1/307; A61B 1/018

(57) ABSTRACT

A surgical method including inserting a puncture wire and sheath in a first direction through a working channel in an ureteroscope and advancing the puncture wire from the sheath into a selected papilla and through a flank of a patient so the puncture wire has an emergent segment extending beyond the flank. A catheter is advanced over the emergent segment of the puncture wire outside the flank in a second direction opposite a direction of renal and flank puncture and into the flank in the second direction, the catheter having a lumen and a radiopaque portion to provide visibility of a tip of the catheter via fluoroscopic imaging. The puncture wire is then removed from the catheter after the tip of the catheter is positioned in a desired region.

### BACKGROUND

This application claims priority to three provisional applications 63/351,463, filed on Jun. 13, 2022, 63/391,771, filed on Jul. 24, 2022 and 63/391,785, filed on Jul. 24, 2022 and is a continuation in part of application Ser. No. 17/860,176, filed on Jul. 8, 2022 which claims priority to provisional application 63/315,578 filed on Mar. 2, 2022, 63/254,101, filed on Oct. 9, 2021, 63/254,128, filed on Oct. 10, 2021, and 63/254,503, filed on Oct. 11, 2021. The entire contents of each of these applications are incorporated herein by reference.

### TECHNICAL FIELD

This application relates to a percutaneous puncture systems and methods for creating a tract for endourologic procedures and to methods and systems to facilitate endourologic procedures after creation of the tract.

### BACKGROUND OF RELATED ART

A nephrostomy creation procedure is the creation of a communication between the skin and kidney to provide for endourologic procedure and therapy. The objective in nephrostomy tract creation is to have the wire from outside the flank directed down the ureter to provide therapeutic endourologic management or treatment of a renal system. The setting of percutaneous endourologic procedures, including but not limited to percutaneous nephrolithotomy, allows for subsequent dilation of the tract, such as by a nephrostomy dilating balloon, between the kidney and the skin over a wire that extends down the ureter. The catheter and tract can also be used to facilitate stenting of a narrowed ureter or removal or treatment of stones obstructing the ureter. Current nephrostomy tract creation is based on x-ray exposure or ultrasound to guide the physician where to locate the nephrostomy puncture wire tract.

Two widely used techniques for nephrostomy tract creation are currently utilized. One technique utilizes an antegrade approach. The antegrade approach holds increased bleeding risk due to the puncture needle puncturing the interlobar arteries as it passes into the collecting system. This antegrade approach is also skill intensive because it requires advancing from the flank to the calyx using two dimensional imaging or targeting aids. In fact, studies have shown that recent urology resident graduates often do not continue to perform the antegrade nephrostomy technique after graduating due to difficulty of this procedure. The fluoroscopy based antegrade puncture procedures require a relatively large amount of radiation exposure.

The other technique commonly utilized is the retrograde puncture technique. This technique is used to create a nephrostomy tract in a retrograde fashion. The original system for this was called the Lawson technique. The Lawson technique is performed under fluoroscopy utilizing a deflecting wire inside a ureteric catheter to select the renal calyx to be entered. That is, fluoroscopy is used to identify the renal calyx for nephrostomy access. The Lawson technique is described for example in Smith's Textbook of Urology, 2007, BC Decker Inc., “Retrograde Access” by Dennis H. Hosking and is commercially available by Cook Urological, Inc. as the “Lawson Retrograde Nephrostomy Wire Puncture Set.”

In the Lawson technique, a stainless steel 145 cm long guidewire (0.038 inches in diameter) having a 3 cm flexible tip is passed retrograde up the ureter into the renal pelvis under fluoroscopy. A 7 Fr catheter is passed over the guidewire into the renal pelvis and the guidewire is removed. A J-tipped wire in certain instances might be used to facilitate passage past an obstruction. Then the surgeon selects the optimal calyx for nephrostomy placement, optimization usually being defined by allowing easiest access to the renal calculi and the shortest tract. Once the calyx is selected, the 0.045 inch diameter deflecting wire guide is inserted through the lumen of the catheter and twist locked to the proximal end of the catheter. Deflection of the wire tip deflects the tip of the catheter, and the catheter and attached wire can be advanced into the selected calyx. However, it is recognized that due to obstructions, e.g., presence of calculi, it may not be possible to advance the catheter into the optimally desired calyx and consequently a less optimal calyx is selected by the surgeon.

After insertion of the catheter into the selected calyx, the deflecting wire guide is removed from the lumen of the catheter, while maintaining the inner-calyx position of the catheter tip. A puncture wire and sheath as a unit are inserted through the catheter lumen, with the puncture wire sharp tip shielded by the sheath. During insertion through the catheter, the wire remains retracted within the sheath, and locked to the sheath by a pin vise lock, so its puncture tip is not exposed. The puncture wire and sheath are connected/locked to the proximal portion of the catheter. The puncture wire is then unlocked from the sheath, by untwisting the cap of the pin vise actuator to loosen the vise pin grip on the puncture wire, and then incrementally advanced from the distal end of the sheath through the flank, fascia and skin. After puncturing the skin, the puncture wire is advanced from below until approximately 15 cm of wire is externally visible.

The pin vise lock securing the puncture wire to the insulating sheath is then re-locked. A fascial incising needle may or may not be passed over the puncture wire at the flank to incise fascia, and is then removed. As the 7 French catheter is advanced through the cystoscope below, the puncture wire is drawn further out of the flank, until the tip of the 7 French catheter is delivered out of the flank. At this time, the 7 French catheter is unlocked from its connection to the puncture wire assembly, and the puncture wire and insulating sheath are removed from below. A 0.038″ guidewire is then passed antegrade through the 7 French catheter from the flank, until it emerges out the lower end of the 7 French catheter at the cystoscope end. With this wire ‘through and through’ the body, the cystoscope and 7 French catheter are removed, leaving the guidewire in place.

The retrograde Lawson approach has several advantages over the antegrade approach including providing the surgeon an anatomic approach to the renal pelvis, increased likelihood of avoiding the interlobar arteries during puncture, and inherently having a wire down the ureter, an important step in securing control over the nephrostomy tract. It is also less skill intensive, due in large part to the fact that it enables travel from the “known kidney” to the “unknown flank/skin,” which better respects the principles of surgery.

However, despite its advantages over the antegrade approach, there are several disadvantages to the Lawson technique. First, it is often difficult to navigate the ureteric catheter beyond large obstructive stones in the renal pelvis. This inability to direct the catheter to the desired site (calyx) often leads the surgeon to access a less optimal calyx. Second, fluoroscopy provides only a two dimensional view of the renal anatomy, thereby limiting the ability to confidently select the calyx for tract dilation. Sometimes, there is even uncertainty as to which calyx is actually chosen due to the limited visibility provided by fluoroscopy.

Consequently, it would be advantageous to provide a system and method that enables more precise calyx location, improves access to the calyx of choice, improves visualization, permits “fluoroscopy-free” calyx selection, and allows for preliminary laser lithotripsy of a portion of a stone that may block access to calyx of choice for nephrostomy creation. Also of significance is that nephrostomy tube creation procedures are usually performed by interventional radiologists, which can further compound the risks and problems since urologists usually have better success rates for selecting the calyx for such procedures. Thus, it would be advantageous if such improved system and method could be more commonly performed by urologists.

In an attempt to address some of the disadvantages of the Lawson technique, Dr. Larry C. Munch in an article entitled “Direct-Vision Modified Lawson Retrograde Nephrostomy Technique Using Flexible Ureteroscope” and published in the Journal of EndoUrology, Volume 3, Number 4, 1989, described a technique utilizing a flexible ureteroscope.

In this “Munch technique,” a flexible steerable ureteroscope was utilized to inspect the renal pelvis and calices. As described, a flexible cystoscopy is performed and a 0.035 inch, 145 cm guidewire is passed into the ureteral orifice. Position within the ureter is assessed with fluoroscopy. The cystoscope is removed and a ureteral access sheath with its obturator is advanced over the guidewire, and the obturator is then removed and the ureteroscope is passed through the sheath into the renal pelvis. An appropriate calyx is chosen visually, and then the 0.0017 inch Lawson puncture wire and protective 3Fr radiopaque Teflon sheath is passed through the working channel of the ureteroscope. The calyx is entered and the sheath embedded in the wall of the calyx, and then the pin-vise lock which locks the puncture wire and sheath together is opened and the puncture wire is advanced through the skin under visual and fluoroscopic control. The puncture wire protective sheath and ureteroscope are then withdrawn, leaving the puncture wire and ureteral access sheath in place. At the skin, an 18 gauge needle is passed over the puncture wire into the kidney and then removed. A 9 French fascial dilator is then passed over the 0.017 inch puncture wire into the kidney, whereafter the puncture wire is removed and a 0.038 inch guidewire is passed through the 9 French dilator until it passes down the ureter through the access sheath, and exits through the urethra. Thus, Munch was the first to describe retrograde nephrosotomy via retrograde puncture from “inside to outside” though a flexible ureteroscope pre-positioned in a renal calyx.

Although the Munch technique solves some of the problems associated with the Lawson technique, it still has several deficiencies. Munch's technique of antegrade wire exchange is ineffectual and risks cutting the puncture wire with passage of 18 gage hollow bore needle over the wire. After passage of this needle, a 9 French fascial dilator is passed over the 0.017″ puncture wire, representing a wire-catheter mismatch which can result in tearing of internal tissues. This large jump from an 18 gauge needle to a 9 French fascial dilator is also cumbersome and has a high chance of failing to grant access to the kidney. In addition to employing a wire that could both kink during regular use and “shear” through the kidney and ureteropelvic junction if exposed due to wire thickness, Munch's wire exchange system has been ineffectual, with a reported use of a rigid needle cannula which could not direct the wire into the ureter.

Consequently, it would be advantageous to provide a system and method that would enable urologists to more economically and efficiently perform the nephrostomy procedure to obtain access for nephrostomy tube creation. Such procedure would have the above-noted advantages over the Lawson technique, e.g., improving calyx access, visualization etc., while also providing the advantages of reducing the number of surgical steps and reducing the possibility of tearing tissue, thereby providing advantages over the Munch technique.

In U.S. Pat. Nos. 8,771,287 and 8,888,787 (common inventor and owner as the present application), systems and methods are disclosed which provide improvements to the foregoing deficiencies and overcome the disadvantages of Lawson, Munch and other prior systems. The systems of these patents use a coaxial catheter to effect wire exchange as well as a dual diameter puncture wire to provide a thicker portion of wire over the ureteropelvic junction if exposed to this anatomic region. These features are incorporated into the commercialized RetroPerc* Flexible Ureteroscopy Guided Retrograde Nephrostomy Wire Puncture Set. In this setting, the puncture wire performs both a) renal puncture and b) serves as a bridge over which the wire exchange catheters are advanced at the flank, where the tip protector sheath over the puncture wire does not emerge from the flank. The RetroPerc* system employs a coaxial microintroducer advanced over the puncture wire at the flank to permit placement of a larger diameter second nephrostomy wire. These systems have several advantages which are described in these patents.

The inventor of these systems recognized that alternative systems could be utilized to achieve the same and in fact additional advantages, and conceived and designed unique variations which enable endourologic procedures. These are described in commonly assigned U.S. application Ser. No. 17/180,176, filed Jul. 8, 2022, the entire contents of which are incorporated herein by reference. The inventor of these systems recognized that alternative systems could be beneficial in certain applications.

The inventor of these systems also recognized that systems to facilitate endourologic procedures after nephrostomy tract creation could be beneficial in meeting the needs in certain clinical applications. Thus, the present inventor developed systems and methods to satisfy the need in the art for improved access to the renal system for performing endourologic procedures after establishment of the nephrostomy tract.

### SUMMARY

The present invention in some aspects provides methods of wire exchange at the flank, over an emergent puncture wire for nephrostomy tube creation. Each of the two systems/methods of the present invention employ single lumen exchange catheters and are described in detail herein. The catheters are configured and dimensioned to facilitate wire exchange and ureter access. One system utilizes a single catheter; another system utilizes two catheters. The catheters are sized to accommodate standard sized puncture wires and endourology working wires, and the methods utilize such sizing to obtain desired access.

The present invention also provides in some embodiments a puncture wire construction which advantageously reduces kinking of the wire. This is discussed in detail below.

The present invention also provides in some aspects improved methods of access to the renal system for endourologic treatment after nephrostomy tube (tract) creation to facilitate the endourologic procedures. The tube creation prior to the treatment can be in accordance with the methods disclosed herein or in accordance with other methods.

In accordance with one aspect of the present invention, a surgical method is provided comprising the steps of:
a) inserting a puncture wire and sheath in a first direction through a working channel in an ureteroscope, the puncture wire having a tissue penetrating tip shielded in a sheath;
b) advancing the puncture wire a first distance from the sheath and into a selected papilla and through a flank of a patient so the puncture wire has an emergent segment extending beyond the flank of the patient;
c) advancing a catheter over the emergent segment of the puncture wire outside the flank in a second direction opposite a direction of renal and flank puncture by the puncture wire;
d) advancing the catheter into the flank in the second direction, the catheter having a lumen and a radiopaque portion to provide visibility of a tip of the catheter via fluoroscopic imaging;
e) removing the puncture wire from the catheter after the tip of the catheter is positioned in a desired region; and
f) advancing an endourology wire in the second direction through the catheter into the patient.

In some embodiments, the puncture wire is slidable within the sheath and releasingly lockingly engageable therein, and the puncture wire is released from the sheath prior to the step of advancing the puncture wire through the flank.

In some embodiments, the endourology wire is advanced through the catheter and terminates in a kidney of the patient; in other embodiments, the endourology wire is advanced through the catheter and terminates in a ureter of the patient.

In some embodiments, the puncture wire has a proximal segment with an outer diameter larger than a distal segment of the puncture wire to add stiffness to enable insertion of the catheter over the puncture wire and reduce or prevent kinking of the wire during advancement of the catheter. In some embodiments, the proximal segment of the puncture wire is flexible to permit endoscope deflection of an endoscope positioned over the puncture wire.

In some embodiments, the radiopaque portion comprises a radiopaque band extending around at least a portion of a circumference of the catheter; in other embodiments, the radiopaque portion comprises a radiopaque filler extending within a wall of the catheter along at least a portion of a length of the catheter.

In accordance with another aspect of the present invention, a surgical method is provided comprising the steps of:
a) inserting a dual function puncture wire having a tissue penetrating tip shielded in a sheath in a first direction through a working channel in an ureteroscope;
b) advancing the puncture wire from the sheath a first distance from the sheath and through a flank of a patient so the puncture wire has an emergent segment extending beyond the flank of the patient;
c) advancing an outer member and dilator having a tapered tip over the emergent segment of the puncture wire outside the flank in a second direction opposite a direction of renal and flank puncture by the puncture wire; and
d) removing the inner dilator leaving a distal end of the outer member in a kidney of the patient.

In some embodiments, the outer member comprises a nephroscope having a working channel for insertion of a surgical instrument for performing an endourologic procedure. In some embodiments, the outer member comprises a sheath, and the method further comprises inserting an endoscope through the outer member, wherein the endoscope has a working channel for insertion of a surgical instrument for performing an endourologic procedure. In some embodiments, the outer member comprises a balloon dilation catheter, and the method includes i) inflating a balloon of the balloon dilation catheter; ii) advancing a sheath over the balloon into the kidney; iii) deflating the balloon; iv) removing the balloon catheter leaving the outer sheath in place; and v) inserting an endoscope with a working channel through the outer sheath into the kidney. In some embodiments, the outer member comprises a catheter having multiple lumens and a mechanism to curl the catheter to secure the catheter in the kidney.

In some embodiments, the dilator is removably positioned within the outer member; in other embodiments the dilator is integral with the outer member and extends distally. In some embodiments, the outer member is an endoscope.

In accordance with another aspect of the present invention, a surgical method is provided comprising the steps of:
a) inserting a puncture wire having a tissue penetrating tip shielded in a sheath in a first direction through a working channel in an ureteroscope;
b) advancing the puncture wire from the sheath a first distance from the sheath and through flank, fascia and fat of a patient so the puncture wire can emerge beyond a skin of the patient;
c) if misalignment of a distal segment of the puncture wire within the fat of the patient is detected, inserting a clamping member through the skin of the patient at a site spaced from an intended or a desired site of puncture wire emergence through the skin; and
d) grasping a distal region of the puncture wire with the clamping member and directing the puncture wire through the skin to complete a retrograde nephrostomy tract creation procedure.

In some embodiments, the clamping member is inserted directly through the skin of the patient.

In some embodiments, the method includes the step of inserting a port through the skin of the patient at a site spaced from the intended or desired site of puncture wire emergence and the step of inserting the clamping member comprises the step of inserting the clamping member through a lumen of the port, the port including illumination and visualization.

In some embodiments, the method further comprises the step of either a) releasing the puncture wire from the clamping member and positioning an exchange catheter over the puncture wire; or b) inserting an endoscope or catheter directly over the puncture wire into the kidney.

Various nephrostomy tube creation methods are also disclosed. In accordance with one aspect of the present invention, a method for creating a tract in retrograde fashion for nephrostomy tube creation is provided comprising the steps of:
a) inserting a puncture wire and sheath in a first direction through a working channel in an ureteroscope to exit the working channel, the puncture wire having a tissue penetrating tip shielded in the sheath;
b) advancing the puncture wire a first distance from the sheath into a selected papilla and through a flank of a patient, the puncture wire having an emergent segment extending beyond the flank of the patient;
c) advancing a catheter over the emergent segment of the puncture wire outside the flank in a second direction opposite a direction of renal and flank puncture;
d) advancing the catheter into the flank in the second direction, the catheter having a lumen having a first section and a second section at a distal tip of the catheter, the first section having a first luminal diameter and the second section having a second lumenal diameter smaller than the first diameter;
e) removing the puncture wire from the catheter; and
f) advancing a second wire in the second direction through the catheter into a ureter of the patient, the second wire having an outer diameter greater than an outer diameter of the puncture wire and greater than the second luminal diameter of the second section of the lumen.

In some embodiments, the puncture wire is slidable within the sheath and releasingly lockingly engageable therein, and the puncture wire is released from the sheath prior to the step of advancing the puncture wire.

In some embodiments, the second wire expands the distal tip of catheter when passed through the second section of the lumen.

In some embodiments, the puncture wire has a proximal segment with an outer diameter larger than a distal segment of the puncture wire to add stability to manual advancement of the catheter and prevent kinking of the puncture wire during advancement of the catheter. In some embodiments, the distal segment has a larger diameter than a distalmost puncture point of the penetrating tip. In some embodiments, the proximal segment is positioned outside an entry point to the ureteroscope when the puncture wire is inserted into the ureteroscope.

In some embodiments, the puncture wire has a distalmost puncture point, a first segment adjacent and proximal of the distalmost puncture point and a second segment proximal of the first segment, wherein the first segment has an outer diameter larger than an outer diameter of the puncture point and smaller than an outer diameter of the second segment to allow flexion of the first segment, and the second segment serves as a bridge over which the catheter is passed and limits kinking of the puncture wire during advancement of the catheter thereover.

In some embodiments, the puncture wire has a proximal segment with an outer diameter larger than a distal segment to prevent kinking during both advancement of the puncture wire through the ureteroscope and advancement of the catheter over the puncture wire in the second direction opposite the direction of advancement through the ureteroscope.

In some embodiments, the second section of the lumen of the catheter has little or zero clearance over the puncture wire.

In accordance with another aspect of the present invention, a method for creating a tract in retrograde fashion for nephrostomy tube creation is provided comprising the steps of:
a) inserting a puncture wire having a tissue penetrating tip shielded in a sheath in a first direction through a working channel in an ureteroscope to exit the working channel;
b) advancing the puncture wire a first distance from the sheath through a flank of a patient so the puncture wire has an emergent segment extending beyond the flank of the patient;
c) advancing a first single lumen catheter over the emergent segment of the puncture wire outside the flank in a second direction opposite a direction of renal and flank puncture and advancing the first catheter into the flank to dilate tissue around the puncture wire, the first catheter having a distal tip having an inner diameter being a zero or close to zero fit over the puncture wire to ease passage into flank tissues;
d) removing the first catheter;
e) advancing a second single lumen catheter over the emergent section of the puncture wire outside the flank and advancing the second catheter into the flank and ureter in the second direction opposite the direction of renal and flank puncture;
f) removing the puncture wire leaving the second catheter in position in the ureter and outside the flank; and
g) advancing a second wire through the second catheter, the second wire having an outer diameter larger than an outer diameter of the puncture wire, wherein the second catheter has an inner diameter larger than the inner diameter of the first catheter.

In some embodiments, the second wire has an outer diameter larger than the inner diameter of the first catheter.

In some embodiments, the second catheter has an inner diameter substantially equal to the outer diameter of the second wire.

In some embodiments, the puncture wire is slidable within the sheath and releasingly lockingly engageable therein and the puncture wire is released from the sheath prior to the step of advancing the puncture wire.

In accordance with another aspect of the present invention, a system for creating a tract for nephrostomy tube creation is provided comprising:
a) an exchange catheter having a tapered end forming a reduced diameter tip region, the tip region expandable by passage of a wire having a larger outer diameter than the reduced diameter tip region;
b) a protective sheath; and
c) a puncture wire slidable within the sheath and releasably lockingly engageable with the sheath, the puncture wire having a puncture tip and a thicker proximal segment at a point of advancement by a clinician above and outside an entry to a working channel of a ureteroscope, the proximal segment thicker than a distal segment adjacent the puncture tip, the thicker segment adding stability to advancement of an exchange catheter thereover and preventing kinking during advancement of the exchange catheter thereover.

In some embodiments, the system further comprises a second wire having an outer diameter larger than an outer diameter of the proximal segment of the puncture wire and larger than an inner lumen of the reduced diameter tip region to expand the reduced diameter tip region when received therethrough.

In some embodiments, the second catheter has an inner diameter equal to or larger than an outer diameter of the second wire.

### BRIEF DESCRIPTION OF THE DRAWINGS

So that those having ordinary skill in the art to which the subject invention appertains will more readily understand how to make and use the surgical systems disclosed herein, preferred embodiments thereof will be described in detail hereinbelow with reference to the drawings, wherein:

FIG. 1 is a perspective view of a first embodiment of the puncture wire, pin vise lock and sheath of the present invention;

FIG. 1A is a perspective view of an alternate embodiment of the puncture wire, pin vise lock and sheath of the present invention;

FIG. 1B is a perspective view of an alternate embodiment of the puncture wire of the present invention having a narrower distalmost segment and a beveled tip;

FIG. 1C is a perspective view of an alternate embodiment of the puncture wire of the present invention having a narrower distalmost segment and a pencil-point tip;

FIG. 1D is a perspective view an alternate embodiment of the puncture wire of the present invention having a pencil point tip;

FIG. 1E is side view of an endourology wire having a larger diameter than the puncture wire of FIG. 1D, the two wires shown side by side;

FIG. 1F is a side view of an embodiment of the single lumen dilator/catheter of the present invention having a tapered tip forming a reduced diameter tip region;

FIG. 1G is a side view of two catheters of different diameters for use in the two-catheter method of the present invention;

FIG. 2 is a side view illustrating the pin vise lock of FIG. 1 for locking the sheath and puncture wire together;

FIG. 3 illustrates initial insertion of a puncture wire and sheath of one embodiment through a working channel of a flexible ureteroscope positioned through the ureter and extending into the kidney, for clarity the ureteral access sheath is not shown;

FIG. 4 illustrates the ureteroscope inserted through a selected calyx and the sheath and puncture wire being advanced in a first direction through the ureteroscope;

FIG. 5 is a view similar to FIG. 4 showing a portion of the ureteroscope of FIG. 1 and showing further insertion of the puncture wire and sheath in a first direction through the ureteroscope with the sheath and puncture wire extending distal of the ureteroscope;

FIG. 5A is a view similar to FIG. 5 illustrating the puncture wire advanced in a first direction through the flank and skin;

FIG. 6 is a view similar to FIG. 3 corresponding to the position of the puncture wire and sheath of FIG. 5A;

FIG. 7 is a close-up view of one embodiment of a locking mechanism for the puncture wire and sheath;

FIG. 8 is a view similar to FIG. 7 showing rotation of the knob to lock the puncture wire and sheath;

FIGS. 9-14 illustrate one embodiment of the method of the present invention using one single lumen catheter wherein:

FIG. 9 is a view similar to FIG. 5A illustrating the puncture wire further extended through the skin;

FIG. 10 is a view similar to FIG. 9 showing initial insertion of the single lumen tapered catheter of FIG. 1F inserted in a second direction (antegrade) over the exposed (emergent) segment of the puncture wire of FIG. 1D;

FIG. 11 is a view similar to FIG. 10 showing insertion of the catheter of FIG. 1F over the puncture wire into the ureter and further showing the puncture wire held by a clamp;

FIG. 11A is a close-up view showing the catheter closely fitting over the puncture wire;

FIG. 12 is a view similar to FIG. 11 showing the puncture wire removed leaving the catheter in place;

FIG. 13 is a view similar to FIG. 12 showing advancement of the larger diameter second (working) wire of FIG. 1E in the second direction (antegrade) through the open end of the catheter exposed at the flank;

FIG. 13A is a close-up view showing the second wire being inserted through the catheter of FIG. 13;

FIG. 13B is a close-up view similar to FIG. 13A showing the catheter tip expanded by passage of the second wire;

FIG. 14 is a view similar to FIG. 13 showing removal of the catheter in a direction opposite the direction of its initial insertion;

FIGS. 15-21 illustrate another embodiment of the method of the present invention utilizing two single lumen catheters wherein:

FIG. 15 is a view similar to FIG. 10 showing initial insertion of the smaller single lumen tapered catheter of FIG. 1G inserted over the exposed (emergent) segment of the puncture wire of FIG. 1D;

FIG. 16 is a view similar to FIG. 15 showing insertion of the catheter in a second direction (antegrade) over the puncture wire into the ureter to dilate the tissue around the puncture wire (the puncture wire held by a clamp);

FIG. 17 is a view similar to FIG. 16 showing removal of the catheter in a direction opposite its direction of insertion;

FIG. 18 is a view similar to FIG. 17 showing advancement of a second larger diameter catheter of FIG. 1G in the second direction over the puncture wire into the ureter;

FIG. 19 is a view similar to FIG. 18 showing removal of the puncture wire leaving the second catheter in place;

FIG. 20 is a view similar to FIG. 19 showing insertion of the second larger diameter (working) wire of FIG. 1E through the second catheter; and

FIG. 21 is a view similar to FIG. 20 showing removal of the second catheter in a direction opposite the direction of its initial insertion to leave the second working/endourology wire in place;

FIG. 22 is a perspective view of one embodiment of a kit of the present invention having a puncture wire within a protective sheath along with the single lumen catheter of FIG. 1F;

FIG. 23 is a perspective view of another embodiment of a kit of the present invention having a puncture wire within a protective sheath along with two of the single lumen catheters of FIG. 1G;

FIG. 24A is a side view of an embodiment of a catheter of the present invention having a radiopaque tip;

FIG. 24B is a side view of an alternate embodiment of a catheter of the present invention having a radiopaque filler;

FIGS. 25A-25C illustrate an embodiment of the method of the present invention utilizing the catheter of FIG. 24A wherein:

FIG. 25A illustrates the catheter being inserted over the puncture wire (shown schematically);

FIG. 25B illustrates the endourology wire being inserted through the catheter after the puncture wire has been removed; and

FIG. 25C illustrates the catheter advanced into the ureter and the endourology wire removed;

FIG. 26 is a side of an endoscope for use with the methods of the present invention;

FIG. 27 is a side view of an embodiment of a puncture wire of the present invention having a stiffer proximal segment;

FIGS. 28A-28F illustrate an embodiment of the method of the present invention utilizing a dual function puncture wire wherein:

FIG. 28A illustrates the ureteroscope positioned in a selected calyx;

FIG. 28B is a close up view of the puncture wire emerging from a working channel of the ureteroscope into the calyx;

FIG. 28C illustrates the puncture wire emerging through the skin;

FIG. 28D is a view similar to FIG. 28C showing the outer sheath and inner dilator inserted over the emergent puncture wire;

FIG. 28E is a view similar to FIG. 28D showing the inner dilator removed leaving the distal end of the outer sheath in the kidney;

FIG. 28F is a view similar to FIG. 28C showing insertion of the endoscope through the outer sheath;

FIGS. 29A-29B illustrate an alternate method of the present invention utilizing a dual function puncture wire wherein:

FIG. 29A shows the nephroscope and inner dilator inserted over the emergent puncture wire; and

FIG. 29B is a view similar to FIG. 29A showing the inner dilator removed leaving the distal end of the nephroscope in the kidney;

FIGS. 30A-30D illustrate an alternate method of the present invention utilizing a dual function puncture wire wherein:

FIG. 30A shows a balloon dilation catheter inserted over the emergent puncture wire;

FIG. 30B is a view similar to FIG. 30A showing inflation of the balloon of the balloon dilation catheter and advancement of a sheath over the balloon in the kidney;

FIG. 30C is a view similar to FIG. 30B showing deflation of the balloon and removal of the balloon catheter leaving the sheath in place; and

FIG. 30D is a view similar to FIG. 30C showing insertion of an endoscope through the sheath;

FIG. 31 illustrates an alternate method of the present invention utilizing a dual function puncture wire showing an endoscope with a tapered tip inserted over the puncture wire;

FIG. 32 illustrates an alternate method of the present invention utilizing a dual function puncture wire showing a catheter with a tapered tip inserted over the puncture wire;

FIG. 33 is a side view comparing a normal path (in dotted line) of the puncture wire with a deviated path (in solid line) of the puncture wire in subcutaneous fat;

FIG. 34 is a view similar to FIG. 33 showing insertion of a clamp through the skin to grab the puncture wire to direct the wire;

FIG. 35 is a side view of an alternate embodiment of the present invention utilizing a multi-function port for insertion of a clamp through the skin to grasp the puncture wire to direct the wire;

FIG. 36 is a view similar to FIG. 35 showing insertion of a clamp (grasper) through the multi-function port to grab the puncture wire to direct the wire;

FIG. 37 is a view similar to FIG. 36 showing removal of the multi-function port and grasper leaving the emergent puncture wire in accordance with an alternate method of the present invention;

FIG. 38 shows an exchange catheter inserted over the emergent puncture wire of FIG. 37 in accordance with an alternate method of the present invention;

FIG. 39 illustrates a scope inserted over the emergent puncture wire of FIG. 37;

FIG. 40 is a side view of an alternate method of the present invention utilizing showing a dilating tip obturator inserted over the puncture wire and through an outer sheath;

FIG. 41A is a close up view showing the distal end of the sheath and obturator;

FIG. 41B is a close up view of the wire extending through the obturator;

FIG. 42 is a side view of an alternate method of the present invention utilizing showing a port with a dilating tip inserted over the puncture wire;

FIG. 43 is a side view of the port of FIG. 42;

FIG. 44 is a side view of an alternate embodiment of the present invention showing a radiopaque skin marker and a radiopaque rib marker releasably securable to the rib;

FIGS. 45A-45C are side views showing an alternate method of the present invention utilizing a dual lumen exchange catheter wherein

FIG. 45A shows the puncture wire exposed and a dual lumen catheter being loaded over the puncture wire;

FIG. 45B shows the puncture wire emerging through a first lumen of the dual lumen catheter; and

FIG. 45C shows a larger diameter nephrostomy wire extending through the second lumen of the dual lumen catheter.

### DETAILED DESCRIPTION OF PREFERRED EMBODIMENTS

The present invention in some aspects provides methods of wire exchange at the flank, over an emergent section of the puncture wire section of the puncture wire extending from the flank and skin. In general, two systems/methods are disclosed, both utilizing a puncture wire and a protective sheath. Each of the two systems employ single lumen exchange catheters and are described in detail below. It should be appreciated that multiple lumen catheters could also be utilized provided they meet the dimensional aspects advantages of the single lumen catheters as used in the methods disclosed herein.

In the first system described herein, a single lumen catheter/dilator is used for insertion of an endourology working wire. This simplifies the components and procedural steps of the surgery. In the second system described herein, two single lumen catheters are utilized: a first catheter dilates the tissue around the puncture wire to ease passage of the second catheter over the puncture wire, the second catheter then provides for passage of the larger endourology working wire. More specifically, in the first system, a single lumen catheter is passed over the emergent puncture wire, followed by removal of the puncture wire and insertion of a larger wire, followed by removal of the single lumen catheter leaving the larger wire in place. In the second system, two single lumen catheters are utilized wherein a first catheter is passed over the puncture wire, followed by removal of the first catheter, then insertion of a second larger catheter over the puncture wire, and then removal of the puncture wire and advancement of a second wire into the second catheter, followed by removal of the second larger catheter, leaving the second wire in place.

The present invention also provides in some aspects in some embodiments a puncture wire construction which advantageously adds stability to advancement of an exchange catheter thereover and/or reduces kinking of the wire. This is also discussed in detail below.

The present invention in some aspects provides accessing as well as selecting a calyx under direct visualization utilizing an ureteroscope in order to create a nephrostomy tract.

In accordance with some embodiments of the present invention, a puncture wire is advanced through a working channel of an ureteroscope which has been passed into the kidney in retrograde fashion. The puncture wire is then deployed from the ureteroscope working channel through a surgeon selected calyx and through the kidney and out the flank and skin in a retrograde fashion. This technique obviates the need for antegrade access to the calyx as antegrade access disadvantageously requires significant technical skill due to limited targeting systems which typically function in two dimensions and creates potential risks for the patient including relatively high radiation exposure. This retrograde approach provides improved three-dimensional endoscopic targeting and protects the renal anatomy to prevent tissue damage during the procedure.

The present invention also provides methods for renal access after nephrostomy tube creation. Various methods for such renal access are described in detail below.

Additionally, the present invention provides various kits containing the components for practicing the various methods disclosed herein.

In the drawings, like reference numerals identify similar or like components throughout the several views.

As used herein the term “proximal” denotes the region closer to the user and the term “distal” denotes the region further from the user.

### Nephrostomy Tube Creation

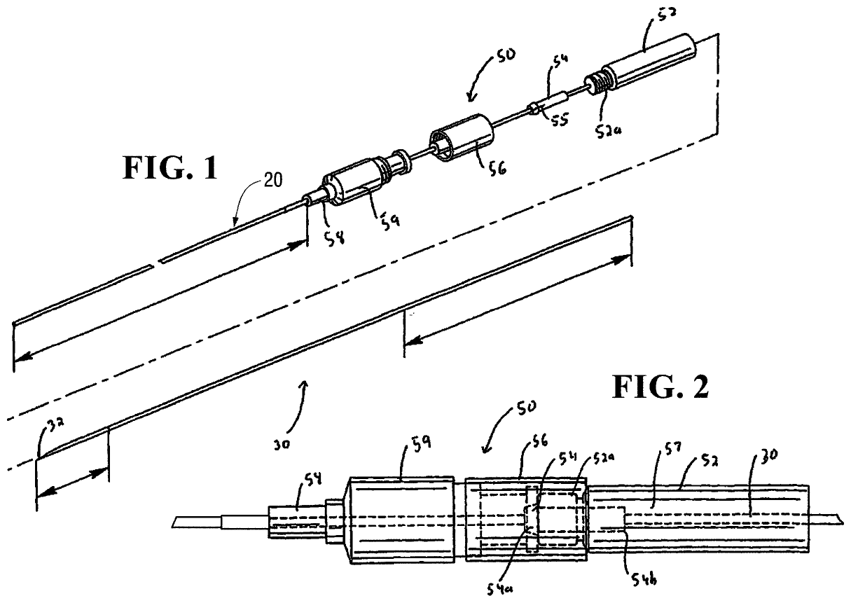

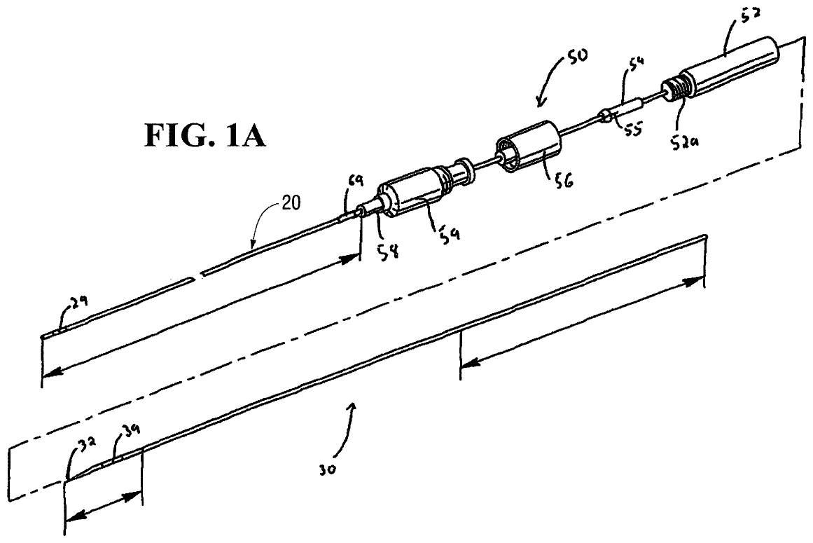

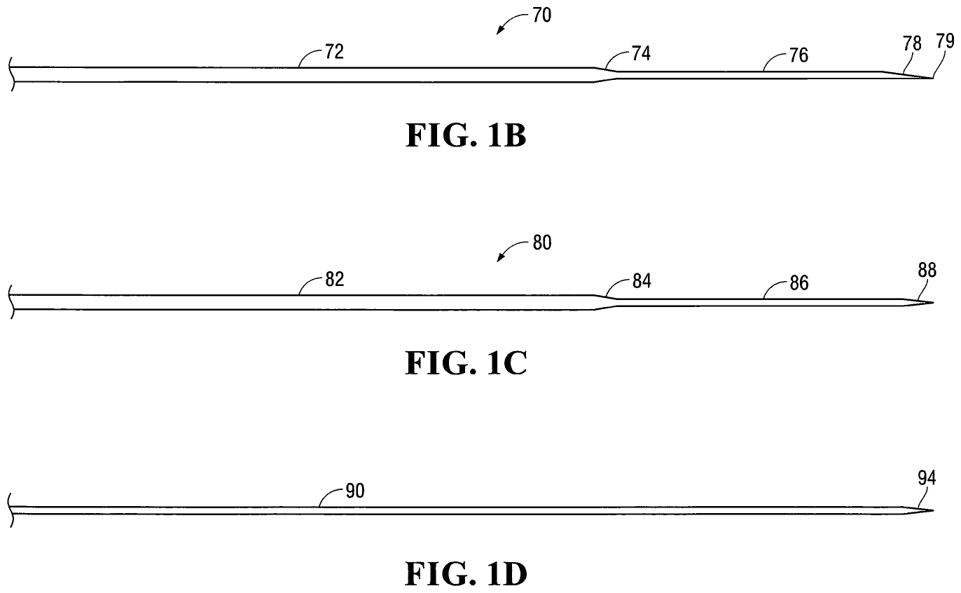

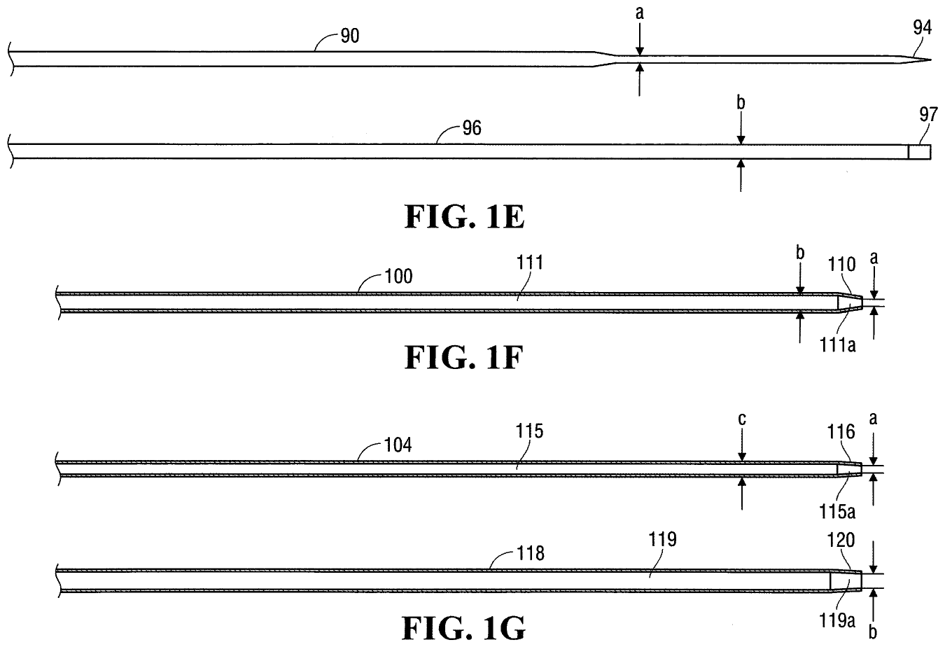

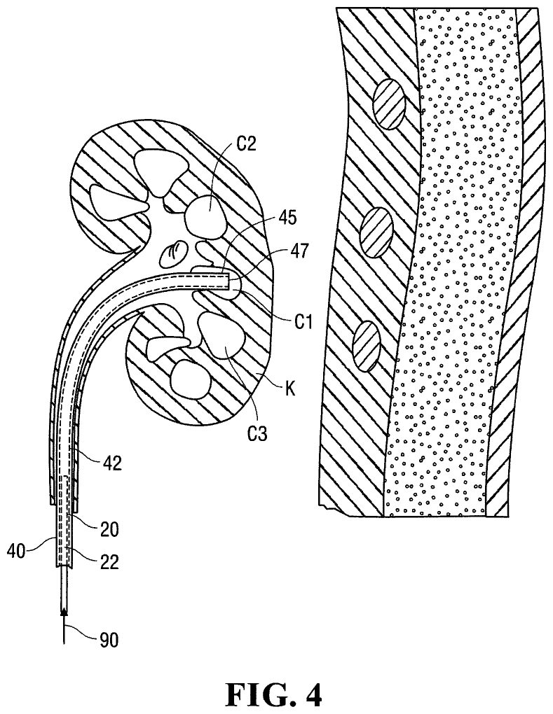

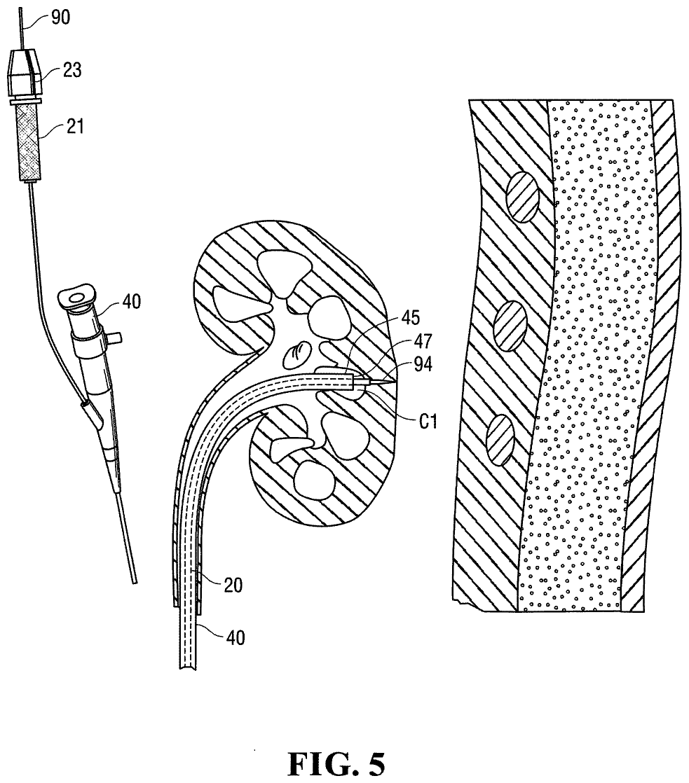

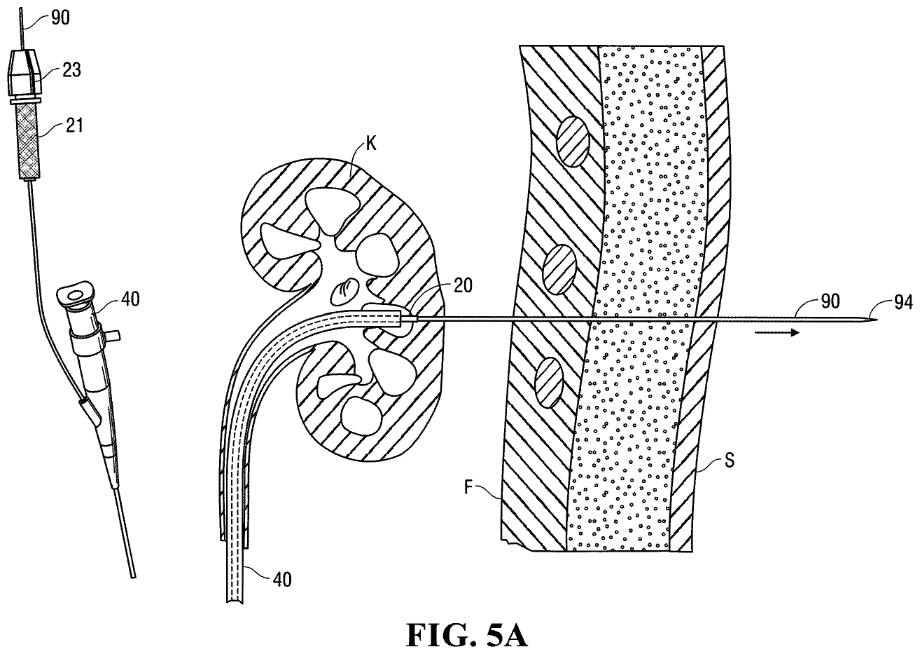

With reference to FIGS. 1, 1A and 2, like the system of U.S. Pat. No. 8,888,787, the system of the present invention includes a protective sheath 20 and a puncture wire 30. Sheath 20 has a lumen 22 extending therethrough dimensioned to slidingly receive the puncture wire 30 therein. That is, puncture wire 30 is received within the sheath lumen 22 for sliding movement from a retracted position wherein the puncture (penetrating) tip 32 of wire 30 is protected (shielded) by the sheath 20 (like the puncture wire of FIG. 4) and an extended position where the puncture tip 32 is exposed from the sheath 20 to penetrate tissue (like the puncture wire of FIG. 5) as the puncture tip 32 extends beyond the distal opening of the sheath 20. Exposure of the puncture wire tip 32 enables advancement of the wire 30 through the flank and skin as described below.

The sheath 20 preferably has a length of between about 70 cm to about 170 cm, and more preferably between about 85 cm to about 115 cm. With this length, the sheath 20 has sufficient length for insertion through the entire working channel 42 of the ureteroscope 40, which typically has a length of approximately 85-95 cm including the portion of channel within the ureteroscope handle. The sheath is preferably a 2.5-2.7 French sheath, having an internal diameter that is sufficient to receive the puncture wire. Other dimensions are also contemplated such as sheath diameters of between about 0.038 inches and about 0.07 inches. The sheath is preferably composed of PTFE (e.g., polyimide or similar), although other materials are also contemplated. The sheath can be constructed with braiding or high durometer materials.

The puncture wire 30 and sheath 20 are releasably locked together by a conventional vise lock 50. FIGS. 1 and 2 show one example of a sheath locking mechanism. Vise lock 50 has a rotatable actuator 52 and a metal locking tube 54 with a longitudinally extending elongated slot. A first (distal) portion 54a of locking tube 54 is seated within tube 56; a second opposite proximal portion 54b is seated within the actuator. Actuator 52 has a reduced diameter portion 52a threadingly received in tube 56 and a lumen 57 through which the puncture wire 30 extends. When actuator 52 is rotated within tube 56, it clamps down on the metal locking tube 54 reducing its diameter due to the slot, to thereby clamp down on the puncture wire 30 to lock it from sliding movement with respect to the sheath 20. A reinforcement tube 58 extends distally from distal tube 59 which can connect via screw threads (or by other methods).

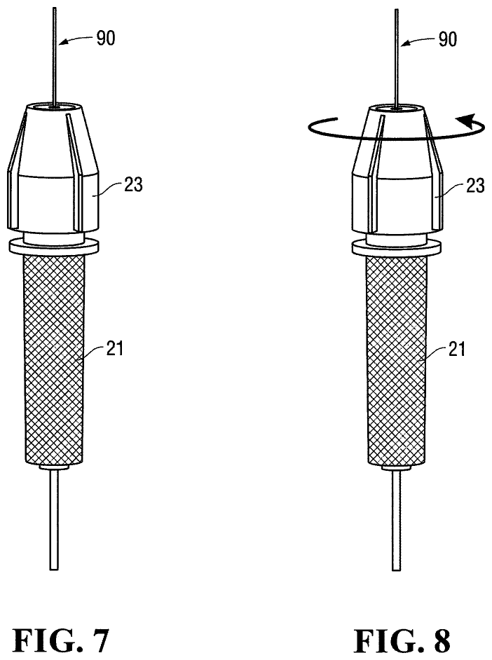

FIGS. 7 and 8 illustrate an alternate embodiment of a pin vise lock for locking the puncture wire and sheath together. Instead of the three piece system of FIG. 1, a two piece system is provided wherein the clamping feature is within tubular portion 21 and rotation of actuator (knob 23), effects clamping down on the puncture wire via clamping down on an internal locking tube similar to tube 54 of FIG. 2. As described below with respect to the method of use, the puncture wire and sheath 20 can be locked together so they can be advanced as a unit through the ureteroscope. When it is desired to move the puncture wire relative to the sheath 20, the actuator 52 or 23 is unscrewed or rotated in a reverse direction, thereby releasing the clamping force on the internal metal tube so the puncture wire can slide relative to the sheath 20.

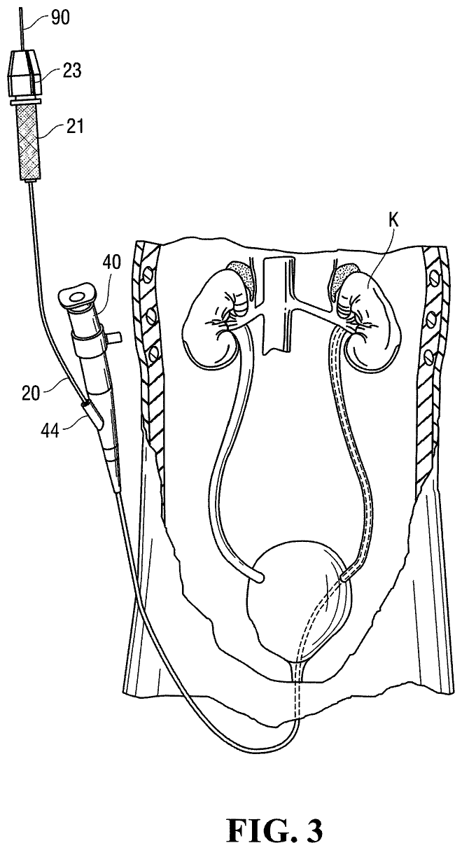

Note the locking mechanism depicted in FIGS. 1 and 2 illustrates puncture wire 30 and the locking mechanism depicted in FIGS. 3, 7 and 8 illustrates puncture wire 90. It should be appreciated that the locking mechanisms disclosed herein, as well as alternative locking mechanisms, can be utilized with any of the puncture wires disclosed herein to lock together the sheath and puncture wire.

The system also can also in some embodiments include a sheath locking mechanism, such as a circumferentially tightening O-ring mechanism for locking the sheath 20 to a working channel of the ureteroscope 40 as described in U.S. Pat. Nos. 8,888,787, and 8,771,287, the entire contents of each of these patents incorporated herein by reference.

The puncture wire 30 of FIG. 1A has a beveled tip. Various alternate puncture wire configurations are disclosed in FIGS. 1B-1D. In FIG. 1D, the puncture wire 90 is similar to puncture wire 30 except it has a pencil point tip 94 rather than a beveled tip. Puncture wire 90 in this embodiment has a uniform diameter along its length, from a proximal end to a distal end except for the distal tip which narrows to form a sharp pencil point tip 94. An increased diameter proximal portion/segment can be provided to reduce or prevent kinking of the portion/segment of the wire outside the ureteroscope during advancement of the wire and at the location of advancement of the exchange catheter.

The puncture wire 80 of the alternative embodiment of FIG. 1C has a pencil-point tip 88 as in wire 90, except it has changing diameters along its length. As shown, it has two diameters, although additional diameters are also contemplated. First, a narrower distalmost segment (portion or region) of the puncture wire 80 has a distalmost puncture point 88. The segment (portion or region) 86 just proximal to the sharp point 88 has a narrower diameter to allow flexion of the distal segment of the flexible ureteroscope while the puncture wire and sheath are inside the working channel of the flexible ureteroscope. The segment (portion or region) 82 of the wire 80 which is proximal to the distal segment 86 has a larger diameter than the distal segment 86. (Reference numeral 84 designates the diameter transition region).

The segment 82 of wire 80 can have a uniform diameter or have an increased diameter at a more proximal region. The segment 82 serves as a bridge over which an exchange catheter is passed. This segment 82 being thicker prevents kinking of the wire 80 during advancement of the exchange catheter from “outside to inside” over the wire. This segment 82 is also thicker than the distal segment 86 of the puncture wire 80 and prevents kinking of the exposed segment above and outside the ureteroscope during advancement. The proximalmost segment of wire 80 can be of the same diameter as the remaining segment 82 or can be of a larger diameter to provide an even thicker kink preventive segment. The proximalmost segment could also in alternative embodiments be a little smaller in diameter that the segment 82 but larger than the diameter of segment 86. The proximalmost segment of the puncture wire 80 is exposed outside the flexible ureteroscope as shown for example in FIG. 3.

FIG. 1B illustrates another alternate embodiment of the puncture wire of the present invention. Wire 70 is similar to wire 80 of FIG. 1C as it has a larger diameter region 72 transitioning at transition region 74 to a smaller diameter region 76. Wire 70 differs from wire 80 of FIG. 1C in that it has a beveled distal end 78 with a sharp point 79 rather than a pencil point. Wire 70, like the other wires disclosed, herein can have proximal segments of increased diameter to reduce kinking.

The puncture wire 90 (as well as the other puncture wires disclosed herein) has a diameter less than a diameter of the endourology working wire, also referred to herein as the replacement wire. This can be appreciated by reference to FIG. 1E showing the puncture wire 90 by way of example, with an outer diameter “a”, next to a working wire 96 with an outer diameter “b” larger than outer diameter “a”. The tip 97 of wire 96 can also have a diameter equal to or larger than diameter “a.” By way of example, diameter “a” could be in the range of about 0.013 inches to about 0.027 inches, and preferably about 0.019 inches, and diameter “b” could be in the range of about 0.025 to about 0.042 inches and preferably about 0.035 inches or 0.038 inches. Other diameters are also contemplated. The diameters of wire 90 are smaller than an internal diameter of the protective sheath 20 to allow sliding movement within the sheath.

It should be appreciated that puncture wire 90 is shown side by side with replacement wire 96 (also referred to herein as the endourology wire, working wire or the second wire) by way of example. The other puncture wires 30, 70 and 80 would also preferably have an outer diameter in the ranges set forth above (diameter “a”) and function in the same manner as puncture wire 90. Also, as should be appreciated, in the methods described below, puncture wire 90 is described and shown by way of example, it being understood that other puncture wires, e.g., puncture wire 30, 70 and 80, can also be used in the same manner as puncture wire 90.

A conventional ureteroscope 40 is shown in FIG. 3 and includes a working (operating) channel opening communicating with an internal channel (lumen) 42 (see e.g., FIG. 4) forming a working lumen for insertion of the puncture wire and sheath. The ureteroscope 40 is preferably a steerable scope so it can be articulated through the urinary system to gain access to the desired calyx. The working channel 42 is accessible through an opening in a port or hub, e.g., through a side arm, which communicates with the ureteroscope channel 42 extending longitudinally within the length of the ureteroscope 40. The ureteroscope 40 provides both illumination and visualization of the surgical site as well as illumination and visualization of the puncture wire and sheath 20 as they are advanced from the distal opening 47 of the ureteroscope 40, thus providing visualization of the system components as well as the patient's body. The ureteroscope 40 typically has a working channel length of about 55 cm to about 75 cm plus a portion of working channel length within the handle of ureteroscope of about 10 cm to about 25 cm (total working channel length about 75 cm to about 100 cm), a total outer diameter at the tip typically of about 5 French to about 8.1 French, with a working channel diameter of about 3 French to about 4.5 French. The working channel 42 is also dimensioned to receive a laser fiber for reducing blocking stones as described below. The ureteroscope is preferably inserted through a ureteral access sheath (not shown).

The puncture wire 90 preferably has a length of between about 130 cm to about 185 cm, and more preferably a length of about 160 cm. With this length, the puncture wire 90 has sufficient length for insertion through the entire working channel 42 of the ureteroscope 40 as well as sufficient length to exit therefrom and extend through the flank and skin. The puncture wire can be composed of stainless steel, although other materials are also contemplated. Note that other wire lengths and diameters are also contemplated.

The puncture wires of the FIGS. 1A-1D can in some embodiments have one or more markings on their outer surface to indicate to the surgeon its position with respect to the sheath 20, skin, and/or ureteroscope 40. The markings can be placed on a region of the puncture wire extending outside the body or alternatively or additionally on a region extending within the body to be imaged by the ureteroscope 40. Likewise, the sheath 20 can have one or more markings on a region outside the body, and/or inside the body where the marking(s) can be visualized by the ureteroscope 40. FIG. 1A illustrates markings 39 on a distal portion of puncture wire 30 and markings 29 on a distal portion of sheath 20 by way of example.

As noted above, there are two different systems/methods of the present invention. One system (referred to herein as the “first” system solely for convenience) includes a single lumen dilator catheter 100 with a lumen 111 as shown in FIG. 1F. At the distal tip 110 of the catheter 100 is a reduced diameter flexible region which has lumen 111a which is smaller than lumen 111. That is, the distal end of lumen 111 tapers in a distal direction to lumen 111a having a smaller diameter. The OD of the distal tip 110 also tapers to a reduced diameter. Stated another way, lumen 111 has a first section and a second more distal section at the distal tip having a smaller diameter than the first section. The first and second sections can each be of uniform diameter or of changing diameters provided the lumen at the distal tip has the smaller diameter than more proximal regions. In preferred embodiments, the inner diameter of catheter 100 is large enough to accommodate a second replacement/endourology/working wire such as wire 96 of FIG. 1E. The outer diameter of the distal tip 110 of the catheter 100 can in some embodiments be smaller than the outer diameter of the second wire 96 to ease passage into the flank tissues over the narrower (smaller diameter) puncture wire.

In the illustrated embodiment, the catheter 100 has an inner diameter “a” at the distal tip which is the same (or substantially the same) as the outer diameter “a” of the puncture wire 90. Catheter 100 has an inner diameter “b” which is greater than the outer diameter “a” of the puncture wire and equal to (or slightly larger than) the outer diameter “b” of working wire 96 as shown in FIG. 1E. In this manner, puncture wire 90 does not stretch the distal tip 110 while working wire 96 does stretch tip 110 due to its smaller inner diameter. Thus, the lumen of the catheter 100 accepts the larger working wire 96 as well as the puncture wire 90, and the distal tip 110 of catheter 100 is tapered to a diameter of the smaller diameter puncture wire such that the puncture wire does not expand/stretch the distal tip 110. Note the puncture wire would fit loosely within the lumen 111 of the catheter 100 proximal to lumen 111a.

In the first system, the single lumen dilator/catheter in preferred embodiments is about 12 cm to about 50 cm in length, and preferably about 20 to about 40 cm. This catheter preferably has an inner diameter of at least 0.025″ and less than 0.055″. It could also have an inner diameter between about 0.035″ and about 0.045″ to accommodate the larger diameter second endourology working wire after removal of the narrower puncture wire from within.

Note FIG. 1F shows a single catheter used in one method of insertion utilizing one catheter. FIG. 1G illustrates two catheters for use in an alternative system/method. This two-catheter system is referred to herein as the “second” system for convenience. The two catheters 114, 118 are of different lumenal and tip diameters. Catheter 114 has a lumen 115 tapering at distal tip 116 to a smaller lumen 115a. Thus, the lumen 115 has a first section and a second more distal section 115a at the distal tip having a smaller diameter than the first section. The internal diameter “a” at tip 116 (lumen 115a) has a diameter matching or substantially the same as the outer diameter “a” of wire 90. Catheter 114 has an internal diameter “c” which is less than the outer diameter “b” of the working wire 96 but greater than outer diameter “a” of the puncture wire 90 (and for puncture wire 70, greater than the outer diameter proximal to segment 76, and for puncture wire 80, greater than the outer diameter proximal to segment 86). Thus, catheter 114 has a snug fit over wire 90 (or wire 30, and for puncture wire 70, a snug fit proximal to segment 76, and for puncture wire 80, a snug fit proximal to segment 86). In other words, the catheter 114 has a lumen large enough to pass over the puncture wire 90 but less than the outer diameter of the working wire 96 so it cannot receive the working wire 96.

The second catheter of the second system, designated by reference numeral 118, has a larger inner diameter and larger outer diameter than catheter 114. Catheter 118 has a lumen 119 at distal tip 120 tapering distally to a reduced diameter lumen 119a at the second section of the lumen at the distal tip. The inner diameter at tip 120, i.e., the diameter of lumen 119a, at the distalmost entry into the catheter 118 has a diameter “b” which matches or substantially matches the diameter “b” of the working wire 96 so the second catheter 118 is large enough to pass over the larger second wire 96.

Thus, in this two-catheter embodiment, while the inner diameter of the first catheter 114 is smaller than the outer diameter of the larger wire 96, the outer diameter of the first catheter 114 is larger than the outer diameter of the second wire 96. This means that the catheter 114 passes snugly over the thin first puncture wire 90 and its outer diameter will stretch fascia larger than the second wire 96 so that when the second catheter 118 is loaded over the puncture wire 90, the clearance/space between the puncture wire outer diameter and the tip inner diameter won't prevent advancement as the tissues will have been stretched already.

Note the size/diameter relationship is discussed above with respect to puncture wire 90, but is also applicable to the other puncture wires disclosed herein.

Turning now to the method of use, and with reference to FIGS. 3 and 4, the ureteroscope 40 is inserted through the ureter U and extends up to the kidney K. This is applicable to both the “first” and “second” methods. The ureteroscope 40 is manipulated under vision so its distal end 45 extends into the calyx of choice, e.g., calyx C1 (FIG. 4). Note the ureteroscope 40 can be articulated into the calyx of choice. Note the methods described below utilizes puncture wire 90; however, it should be appreciated that other puncture wires, e.g., puncture wire 30, 70 or 80, can also be used in the same manner as puncture wire 90 and thus the discussion below of the methods of use are fully applicable to use with puncture wires other than the illustrated puncture wire 90.

If during insertion of the ureteroscope 40 a stone is encountered under visualization that is blocking the path to the desired calyx C, e.g., calyx C1, C2, C3 etc., a laser fiber (not shown) can be inserted through the working channel 42 of the already positioned ureteroscope 40 to perform laser lithotripsy to reduce the size of the stone to allow access by the ureteroscope 40 to the desired calyx. The laser fiber can then be removed from the working channel 42.

After placement of the ureteroscope 40 at the desired location, e.g., into calyx C1 of FIG. 4, the puncture wire 30 and sheath 20, locked together by tightening of the pin vise lock mechanism 23 via rotation of knob 21 as described above, are inserted through the working channel 42 of ureteroscope 40. This initial insertion is illustrated in FIGS. 3 and 4. At this point, the puncture wire tip 94 of puncture wire 90 is retracted and thereby shielded within the protective sheath 20.

The puncture wire 90 and sheath 20 are then advanced in the first direction through the working channel exiting just distal of the tip 45 of the ureteroscope 40 (beyond distal opening 47 as shown in FIG. 5), and viewed to make sure they are in the desired anatomical position.

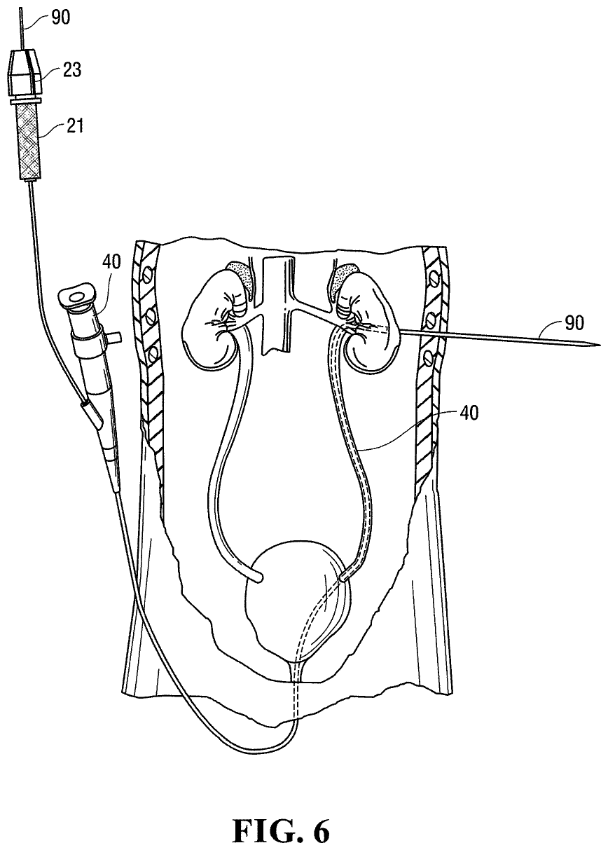

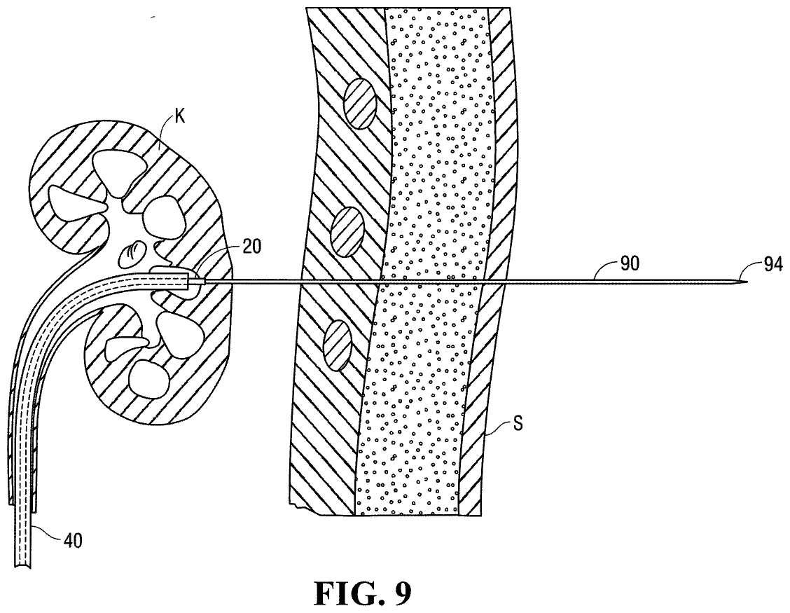

To next advance the puncture wire 90 further through the scope 40 and sheath 20, actuator 23 of pin vise lock 21 is rotated as described above, thereby releasing the locking engagement of the puncture wire 90 and sheath 20. This enables advancement of the puncture wire 90 (see arrow in FIG. 5A) through the kidney K, selected papilla, flank F and skin S as shown in FIGS. 5A and 6. The puncture wire 90 is advanced further in the first direction (retrograde) to a position shown in FIG. 9, with the sheath 20 remaining in the position of FIGS. 5A and 6 and an “emergent” section of the puncture wire exposed. Note that the puncture wire 90 is protected along its length by the sheath 20 as well as by the ureteroscope 40 during insertion, prior to exposure from the sheath in FIG. 5A.

It should be appreciated that alternatively the sheath 20 and puncture wire 90 can be locked together by the pin vise locking mechanism 50, with the puncture tip 94 slightly protruding from the sheath 20, and advanced together through the flank tissues rather than only the puncture wire 30 being advanced through the flank tissues.

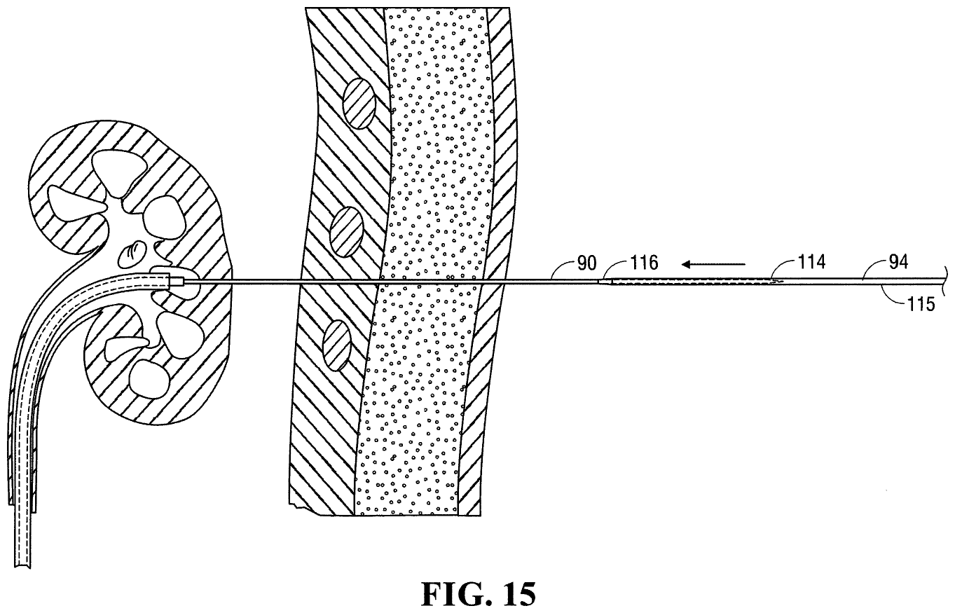

At this point, one of two methods/systems for insertion of the working wire can be utilized. One method is shown in FIGS. 9-14; an alternate method is shown in FIGS. 15-21. For convenience, the methods of FIG. 9-14 will be referred to herein as the “first” method and the method of FIG. 15-21 will be referred to as the “second” method.

The first method is a single catheter system and utilizes catheter 100 of FIG. 1F. As explained above, catheter 100 has a lumen 111 to receive the puncture wire 90. Catheter 110 (referred to herein as C1) as explained above has a distal tip 110 of a reduced diameter as it tapers inwardly in a distal direction to a diameter which preferably matches the outer diameter (OD) of the puncture wire. Stated another way, the smallest diameter of the lumen of the catheter 100 (which is at the distalmost tip) matches/equals (or alternatively is slightly larger than) the OD of the puncture wire 90; the diameter of the lumen proximal of lumen region 111a is greater than the OD of the puncture wire 90:
C1 lumen diameter at tip=OD puncture wire
C1 lumen diameter proximal of tip>OD puncture wire

In this way, the catheter 100 can be easily slid over the puncture wire and the tip of the catheter is not expanded during insertion of the puncture wire. The catheter tip has zero or close to zero clearance over the puncture wire. The narrower tip permits easy passage of the catheter tip through the flank fascia and renal capsule over the puncture wire, and the larger inner diameter (FIG. 1F) accommodates the larger diameter second wire.

The diameter of the lumen of catheter 100 closely matches (is substantially equal) the OD of the working (urology) wire 96, except at the tip:
C1 lumen diameter at tip<OD working wire
C1 lumen diameter proximal of tip=OD working wire

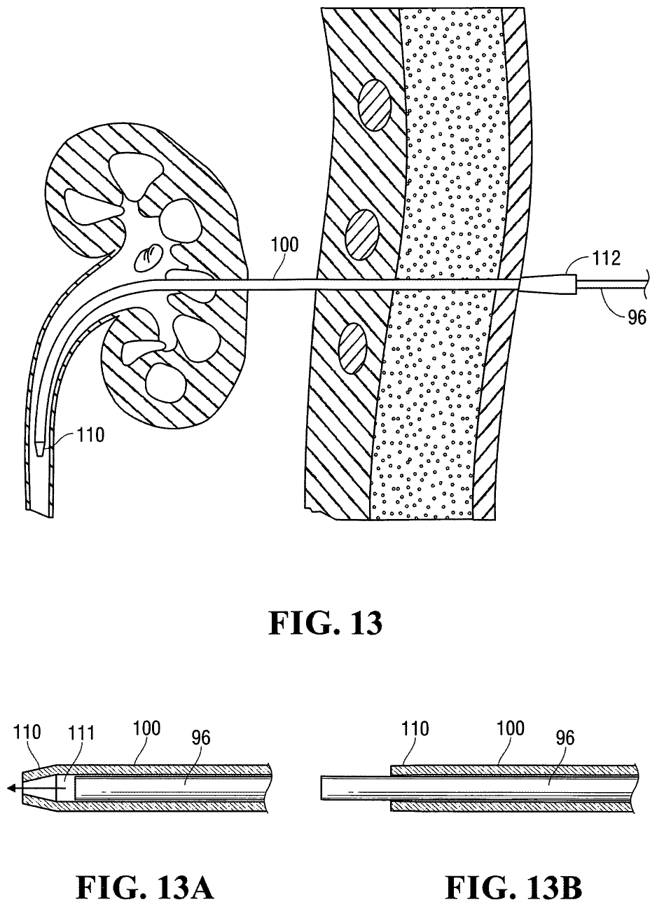

Therefore, when the urology wire 96 is inserted through the catheter 100, it will expand (dilate) the tip as described below in reference to FIGS. 13A and 13B.

The second method utilizes two catheters as shown in FIG. 1G. The first catheter 114 (referred to herein as CT1) has a lumen larger than the OD of the puncture wire and a lumen at the tip matching the OD of the puncture wire:
CT1 lumen diameter at tip=OD puncture wire
CT1 lumen diameter proximal of tip>OD puncture wire

In this manner, the puncture wire does not expand the distal tip of the first catheter 114.

However, the lumen of the first catheter 114 (CT 1) is less than the OD of the working wire 96:
CT1 lumen diameter at tip<OD working wire
CT1 lumen diameter proximal of tip<OD working wire

In this manner, the first catheter 114 cannot receive (pass over) the working wire.

The second catheter 118 (referred to herein as CT 2) of the second system has a lumen equal to the outer diameter of the working wire 96:
CT2 lumen diameter=OD working wire

In this manner, the second catheter 118 can receive the working wire 96.

Note CT2 can have in alternate embodiments a tapered tip less than the OD of the working wire which would be expanded by the working wire. In other embodiments, the lumen at the tapered tip and proximal of the tapered tip is greater than the OD of the working wire.

Turning to the first method of FIGS. 9-14, which utilizes the single lumen catheter, in the next step after the step in FIG. 6 where the puncture wire is advanced through the skin, the puncture wire 90 is extended further from the skin as shown in FIG. 9.

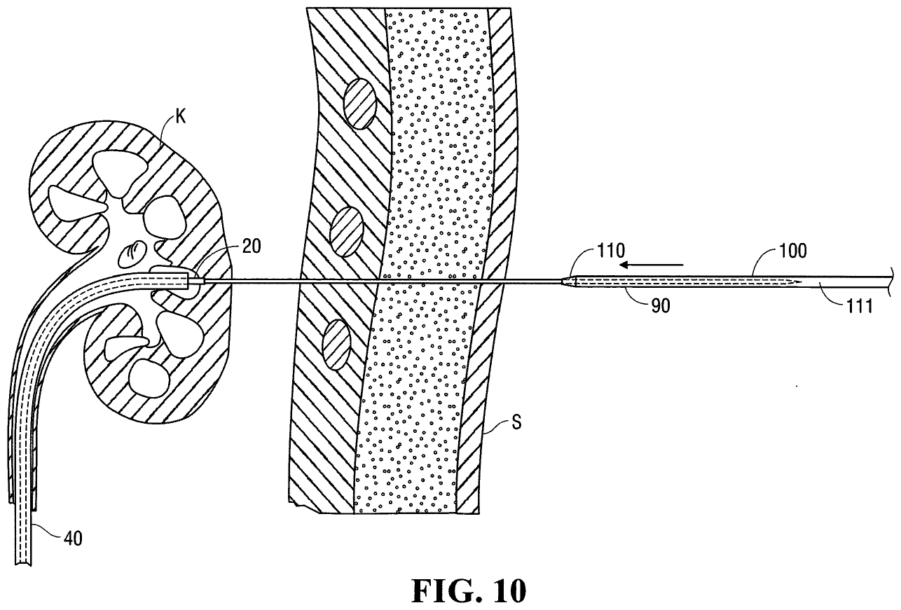

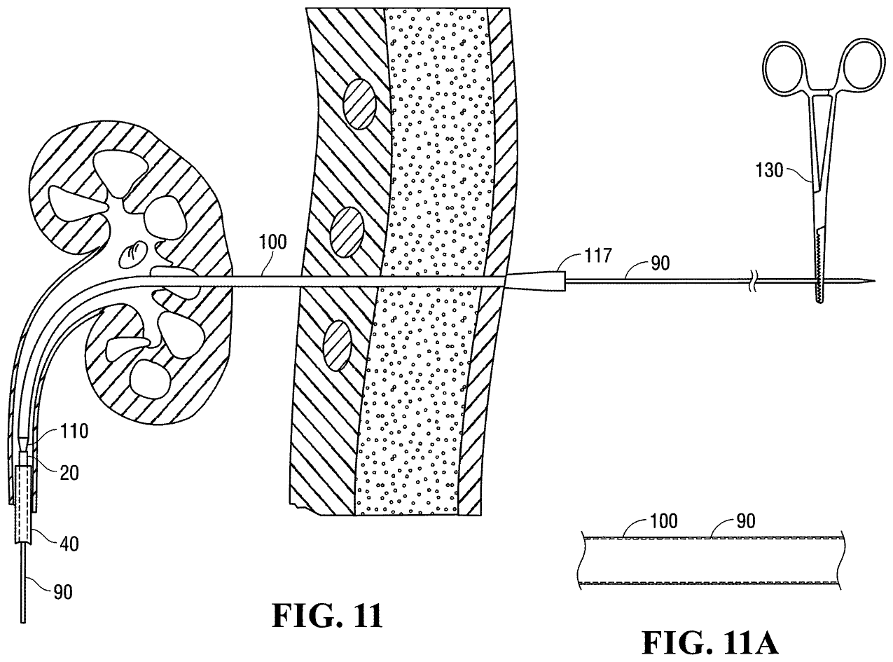

In the next step, illustrated in FIG. 10, the single lumen dilator/catheter 100 is inserted over the emergent section of the puncture wire 90 in a direction opposite to the direction of renal and flank puncture of the puncture wire 90. That is, the catheter 100 is inserted over the puncture wire tip and advanced through the skin in the antegrade direction of the arrow of FIG. 10. The tip of the catheter 100 as noted above is tapered to a diameter narrower than its lumenal diameter to have little or zero clearance over the puncture wire 90 (see FIG. 11A). The narrower tip permits easier passage of the catheter tip through the flank fascia and renal capsule over the puncture wire 90, and the larger inner diameter accommodates the larger diameter second wire inserted in subsequent steps. As shown in FIG. 11, the distal end of the puncture wire 90 exposed from the flank is held in position by a clamp 130 as the catheter 100 is advanced into the ureter.

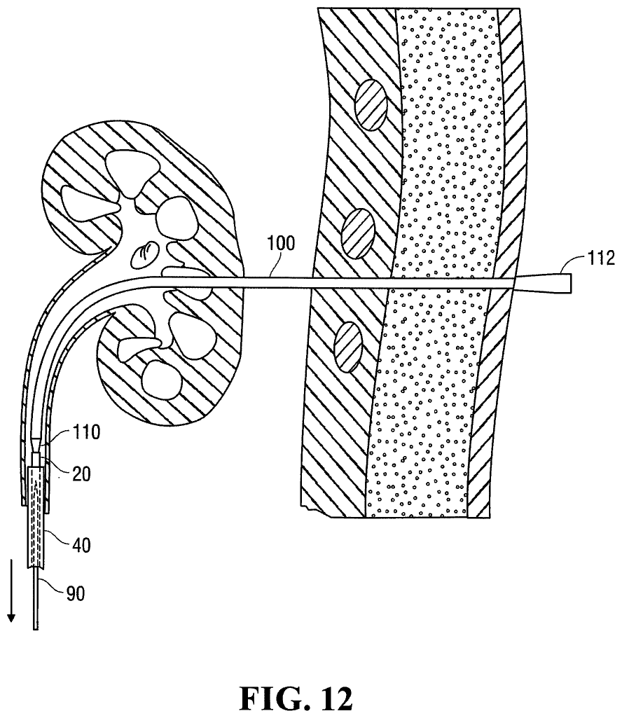

After the catheter 100 is advanced far enough over the puncture wire 90 into the patient to be positioned in the ureter as shown in FIG. 11, the puncture wire is removed (FIG. 12) in the opposite direction of its insertion. The ureteroscope 40 and sheath 20 are also removed in the direction opposite the direction of insertion. Note the tip of the catheter 100 can abut the sheath and/or scope distal edge as it is advanced. The tip of the catheter may enter the open end of a ureteral access sheath which may be positioned in the ureter. Next, the larger diameter endourology working wire 96, is advanced through the open end of the catheter 100 exposed at the flank (FIG. 13), through the length of the catheter 100, and through the narrower tip, dilating the catheter tip 110 as the wire 96 is advanced through the tip 110 as shown in FIG. 13B. Note an acceptably low amount of force is required for advancing the second wire 96 to overcome the narrowed tip 110 of the catheter, thus allowing the larger wire to pass through this tip. Note the scope 40 and sheath 20 are removed prior to advancing the working wire 96 through catheter 100.

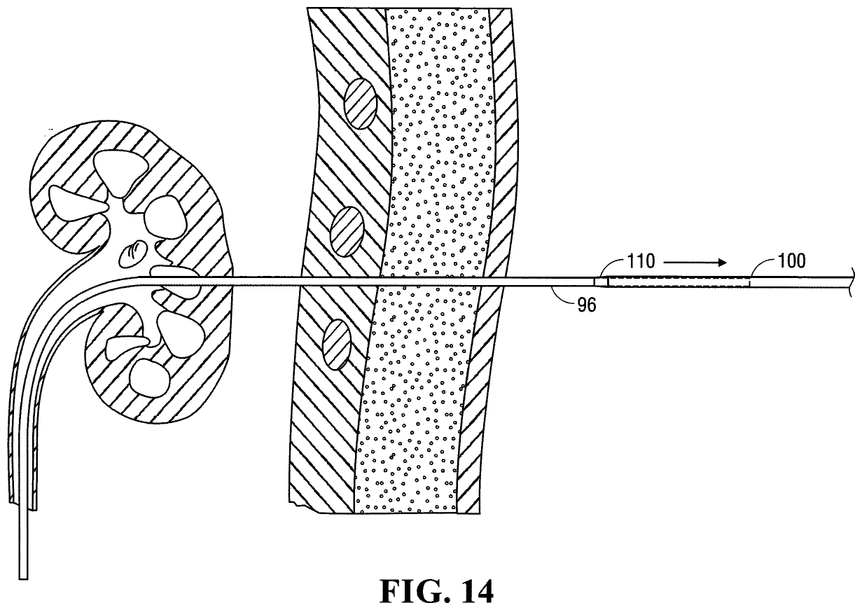

Next, the catheter 100 is removed in the direction opposite the direction of its insertion (in the retrograde direction of the arrow of FIG. 14), leaving the endourology wire 96 in the body extending into the ureter.

An alternate embodiment of the “wire exchange” system and method of the present invention is illustrated in FIGS. 15-21. In this embodiment, the system includes two catheters, preferably single lumen catheters. The method steps of FIGS. 3-6 are the same for this second method and differ from the first method starting at FIG. 15.

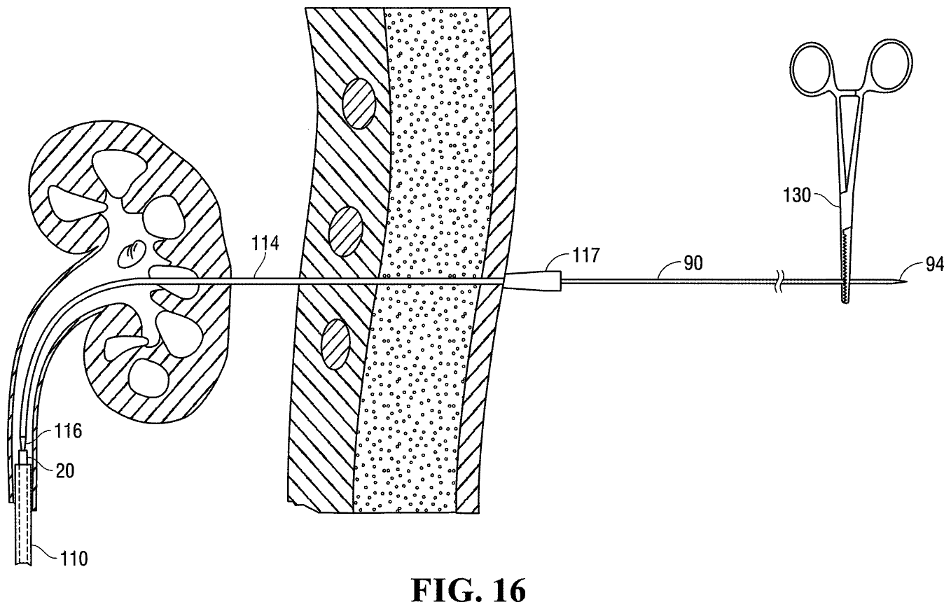

In FIG. 15, the first catheter 114 of FIG. 1G is advanced over the emergent section of the puncture wire 90 at the flank and advanced antegrade into the kidney and ureter for the purpose of dilating the tissues it passes through around the puncture wire (FIG. 16). Thus, its outer diameter is enough to sufficiently dilate the tissues around the puncture wire 90 to ease loading/passage of the second single lumen catheter over the puncture wire whose larger tip diameter is configured to accommodate the second larger diameter wire 96. The catheter tip can abut the tip of the scope and/or sheath during insertion.

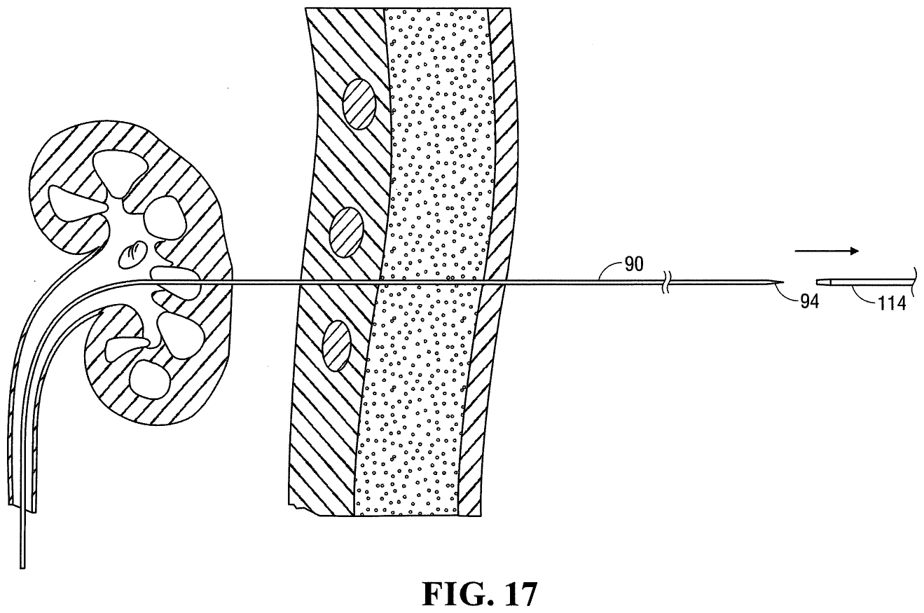

The first catheter 114 is then removed in a direction opposite its direction of insertion (FIG. 17), the second catheter 118 of FIG. 1G is loaded over the puncture wire 90. As noted above, the second catheter has an inner diameter along its length and tip large enough to accommodate the second, larger diameter nephrostomy working wire 96, typically 0.035″ or 0.038″, but at least 0.025″.

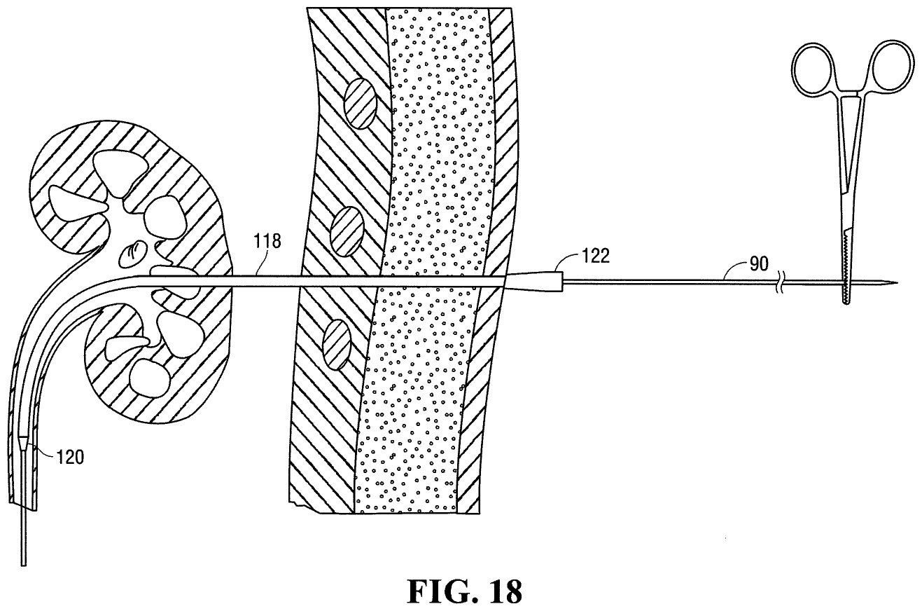

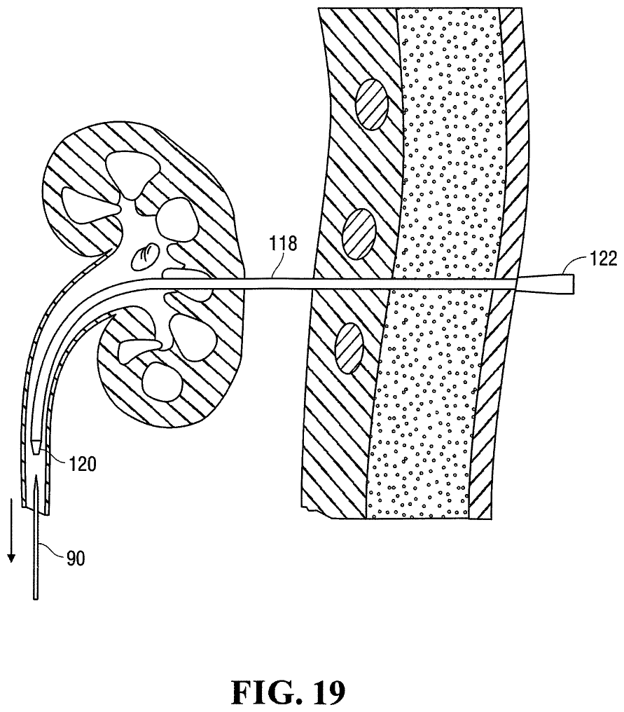

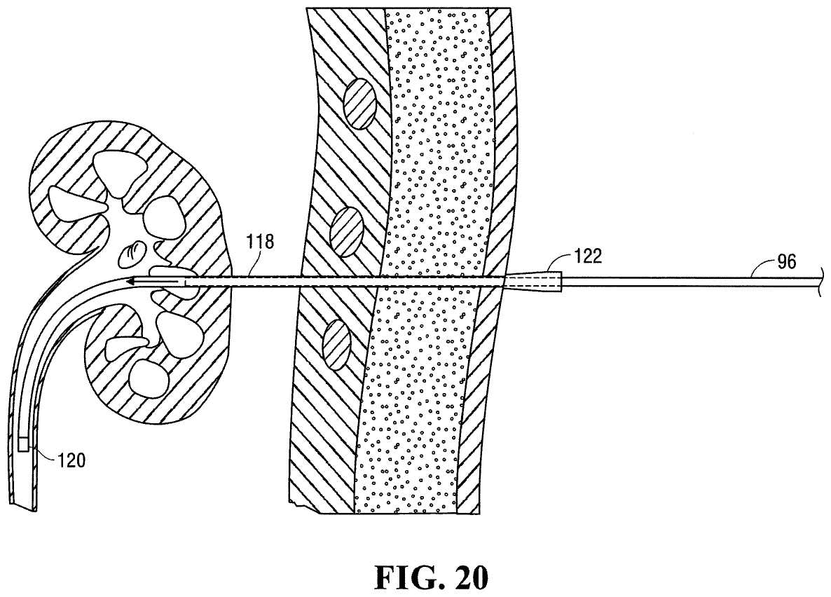

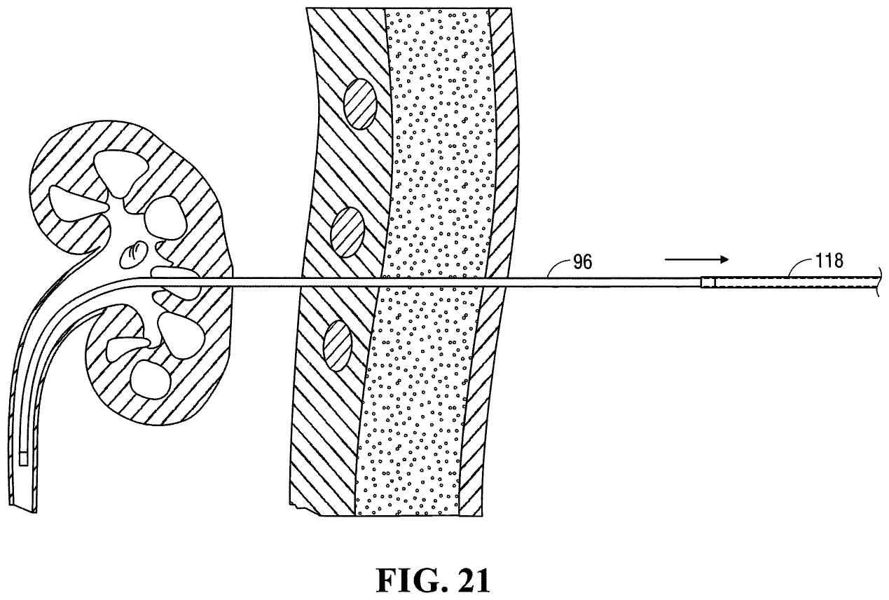

Once the second catheter 118 is advanced antegrade into the ureter (FIG. 18), the puncture wire 90 is removed in the opposite direction of its insertion (FIG. 19) and the second larger diameter working/endourology wire 96 is advanced through the open end of catheter 118 exposed at the flank, through the catheter (FIG. 20), and down the ureter. Note the scope 40 and sheath 20 are removed prior to advancing the working wire 96 through catheter 118. After insertion of wire 96, the second catheter 118 is removed in the direction opposite the direction of insertion as shown in FIG. 21, leaving the working wire 96 in position.

It is also contemplated that in an alternate embodiment, the puncture wire can be utilized without a protective sheath and inserted directly into the ureteroscope 40. The working channel 42 of the ureteroscope in this embodiment would thereby protect the puncture wire during insertion. This would reduce the number of components. Such sheathless puncture wire can be utilized with either method disclosed herein.

In these sheathless embodiments, the puncture wire can be locked to the operating (working) channel 42 of the ureteroscope 40 during insertion of the ureteroscope 40 into the calyx, and then the puncture wire released from locking engagement with the ureteroscope 40 to enable advancement distal of the end of the ureteroscope through the flank and skin. Such locking can be achieved with a vise lock or a locking mechanism similar to locking mechanism 60 described above, with the O-ring clamping on the puncture wire. Such embodiments enable a larger diameter puncture wire to be utilized, which could enable passage of a dilation balloon or other treatment devices directly over the puncture wire, thereby obviating the need for an exchange catheter and a second wire.

It is also contemplated that the characteristics of the puncture wire can be altered. For example, a coating can be applied to improve lubriciousness, and such coating can extend on a portion of or the length of the wire proximal of the tissue puncturing region. Coating with a low friction coefficient material could increase the wire caliber without significantly changing its handling properties. Preferably, the coating would not be applied to the distal 20-30 cm of the wire that is used to puncture the kidney, flank and skin.

The protective sheath for the puncture wire may be constructed to be thin walled to permit the entire puncture wire/protective sheath duo to maintain a small enough total diameter for passage through the working channel of ureteroscope. Use of materials such as PTFE (Teflon) or polyimide for sheath construction may have beneficial properties for this application.

The sheath may be constructed or post-processed to have enhanced visibility under ultrasound imaging. This may be achieved by any number of techniques, which may include but are not limited to placing a ceramic, graphite, Teflon, tungsten, Nitinol or platinum tip or outer coating on all or part of sheath or creating with or post-processing the sheath using laser or other abrasing or cutting technology to create small or microscopic grooves or indentations/dimples in the outer surface of the sheath to increase echogenicity.

It is also contemplated that all or part of the puncture wire and/or the exchange wire may be designed to have enhanced ultrasound visibility. This may allow for reduced radiation exposure during nephrostomy creation by allowing ultrasound guided confirmation of wire location during deployment. Options to achieve this include, but are not limited to the following: 1) Constructing the puncture wire and/or the exchange wire entirely of, or with a component of, a highly ultrasound-visible metal or other material. Examples include, but are not limited to, cobalt/chromium, graphite, Teflon, platinum or tungsten. These components may be mixed with stainless steel as an alloy or simply the distal tip of the wire can be made of these materials. 2) Coating the puncture wire and/or exchange wire with ceramic material, graphite, Teflon, tungsten, platinum, other metals or polymers, or material impregnated with microbubble technology such as glass microspheres, air microbubbles, or other adherent echogenic polymeric films. 3) The puncture wire and/or exchange wire may be constructed with or post-processed to create uneven surface(s) such as by brushing, lasering, creating indentations or cutting the outer surface of the wire. This would increase echogenicity of the wire.

Note the distal tip of the catheters can be radiopaque for localizing by fluoroscopy. To enhance imaging, additional regions of the catheter can be composed of radiopaque material, and even the entire length of the catheter.

The catheter may be constructed or post-processed to have enhanced visibility under ultrasound imaging. This may be achieved by any number of techniques, which may include but are not limited to placing a ceramic, graphite, Teflon, tungsten, Nitinol or platinum tip or outer coating on all or part of the catheter or creating with or post-processing the catheter using laser or other abrasing or cutting technology to create small or microscopic grooves or indentations/dimples in the outer surface of the catheter to increase echogenicity.

To create a nephrostogram to identify renal pelvis anatomy during balloon dilation of nephrostomy tract, the ureteral safety guidewire placed during the ureteroscopy portion of the procedure can be exchanged for a ureteric catheter through the urethra. Retrograde nephrostogram is performed through the ureteric catheter. A Foley catheter is placed in the bladder.

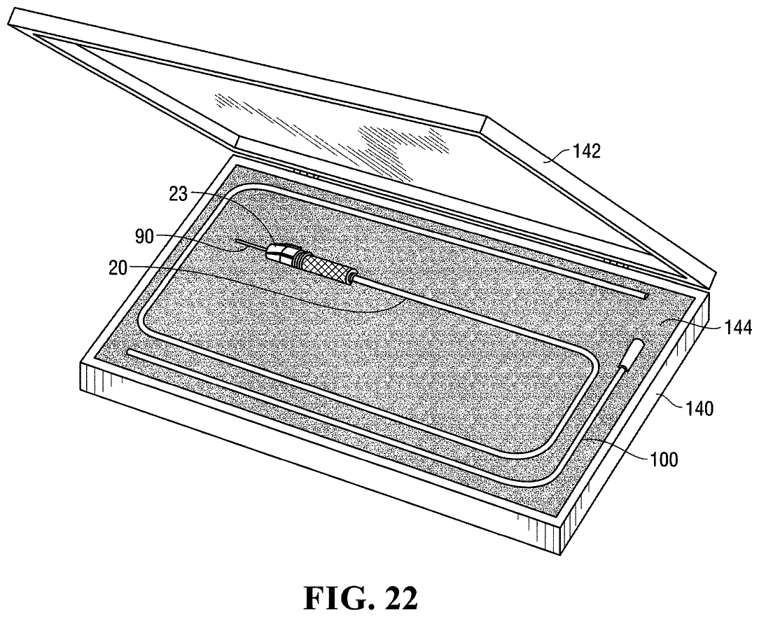

FIG. 22 illustrates one embodiment of a kit of the present invention. In this embodiment, kit 140 includes a cover 142 and packaging 144 with portions to receive the puncture wire 90/sheath 20 assembly as well as a space to receive the catheter 100. This kit can be utilized for the system/method utilizing a single catheter as in FIGS. 9-14. Also shown, the sheath/puncture wire length preferably is 2-3× the length of catheter 100. Note the kit could alternatively include any of the other puncture wires disclosed herein.

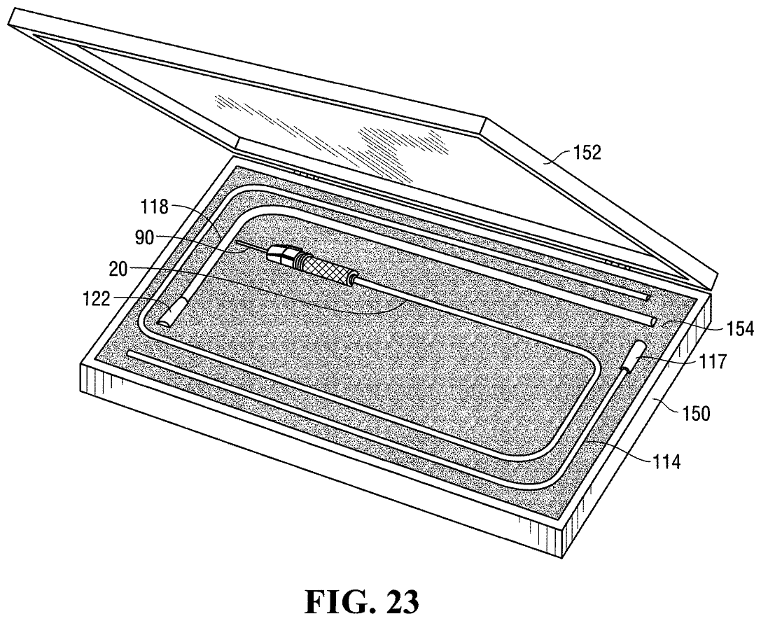

FIG. 23 illustrates an alternate embodiment of a kit of the present invention. In this embodiment, kit 150 includes a cover 152 and packaging 154 with portions to receive the puncture wire 90/sheath 20 assembly as well as a space to receive the catheter 114 and catheter 118. This kit can be utilized for the system/method utilizing two single lumen catheters as in FIG. 15-21. Note the kit could alternatively include any of the other puncture wires disclosed herein. As can be appreciated from the components in kit 150, the sheath/puncture wire 20 can have a length 2-3X the length of the catheters.

The sheath and catheters of kits 140 and 150 could also be packaged in a different configuration than the illustrated L-shape and in different locations than that shown.

### Exchange Catheter System

In the setting of retrograde nephrostomy creation for endourologic surgery the retrograde nephrostomy wire is typically deployed through a flexible ureteroscope positioned in a renal infundibulum, with the ureteroscope tip facing the renal papilla. The puncture wire travels straight, through the renal capsule, perinephric fat, and flank musculature.

After successful retrograde nephrostomy renal puncture with the puncture wire tip emergent from the flank skin, typically the segment of the puncture wire that bridges the outside flank skin to the kidney is not optimal to effect the subsequent endourologic surgery; typically the puncture wire segment is of smaller diameter and more prone to kinking than a standard endourology working wire. The reason this is not optimal is that advancement of instrumentation over this emergent wire in the direction opposite to the puncture may cause a kink of the wire serving as a guide/bridge to the kidney, or ‘slicing’ of kidney/fascia if tension is applied to the emergent wire tip during advancement of instrumentation.

Upsizing and exchange of the puncture wire for a standard endourology working wire in accordance with devices and methods of the present invention is described above, wherein an exchange catheter (or two catheters in the two-catheter systems) is advanced (in a direction opposite the retrograde nephrostomy puncture) over the emergent puncture wire at the flank into the kidney and possibly the ureter. After removal of the inner puncture wire and possible an inner dilator of the exchange catheter (if a coaxial catheter system is used), a new endourology working wire is next advanced into position in the collecting system.

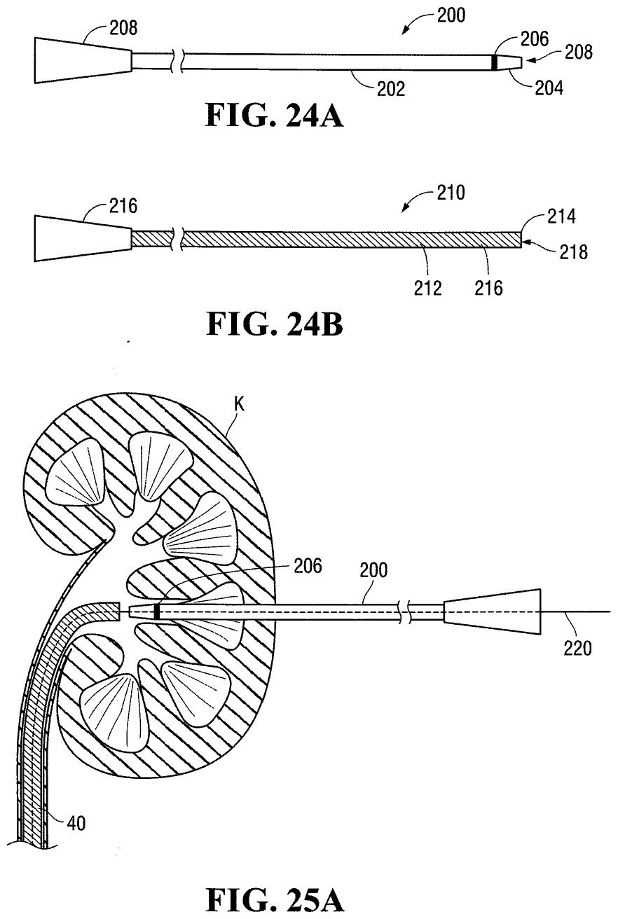

In an embodiment of the present invention, a modification to the exchange catheter system (whether a single catheter system or a coaxial catheter system is used) is provided to create a radiovisible tip. With reference to FIG. 24A, in one embodiment, catheter 200 has an elongated body portion 202 extending from hub 208 and terminating at a distal end in tapered tip 204. The body portion 202 and tapered tip 204 have a lumen extending therein terminating in distal opening 208. A radiopaque segment 206, such as a band, extends around a partial or full circumference of the catheter 200, and can be made of platinum or other radiopaque materials. The radiopaque segment 206 is provided at the end of the catheter 200, either at a distal region slightly proximal of the distal tip 204 as shown in FIG. 24A or alternatively on the tapered tip 204 itself. Thus, the location of the catheter distal tip can be identified by the clinician.

In the alternate embodiment of FIG. 24B, catheter 210 has a radiopaque filler (doping) 216 in the catheter material along a partial or full length of the catheter, i.e., the catheter is formed so the filler is embedded in the wall, so that the catheter along its partial or entire length is highly radiovisible to enable the clinician to know the location of the catheter and tip. Catheter 210, like catheter 200, has an elongated body portion 212 extending from hub 216 terminating at a distal end in tapered tip 214. The body portion 212 and tapered tip 214 have a lumen extending therein terminating in distal opening 218. Catheter 210, like catheter 200, is dimensioned to be inserted over the puncture wire in a direction opposite the direction of puncture wire advancement through the skin.

This ability to discern clearly the tip of the exchange catheter 200, 210 advantageously allows the surgeon to determine where desired to deploy the new, second endourology wire in the renal portion of the collecting system to avoid positioning the second wire in the ureter, or in the ureter itself.

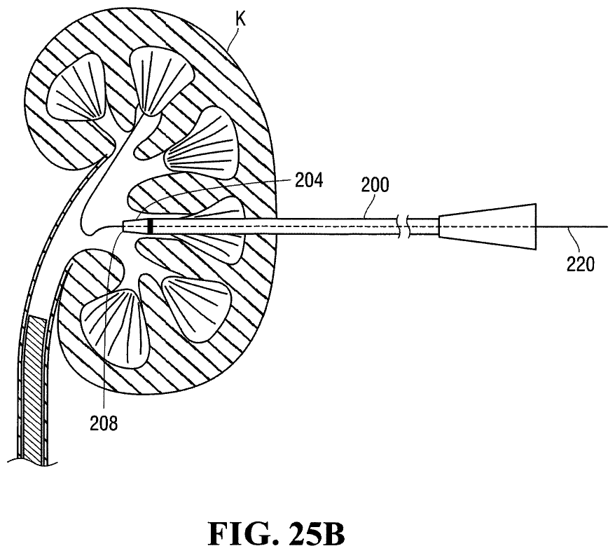

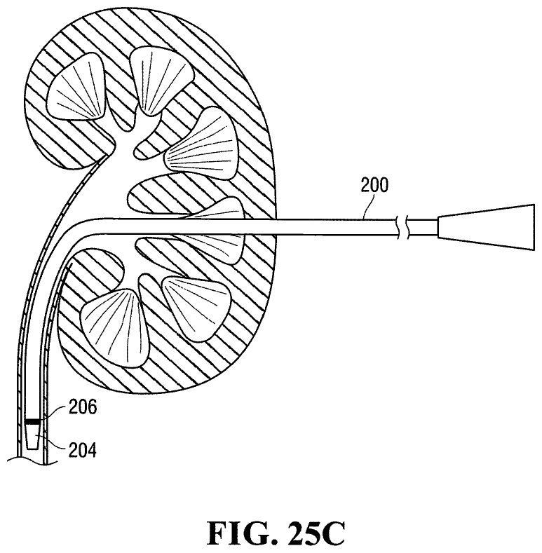

More specifically, as shown in FIG. 25A catheter 200 (or alternatively catheter 210) is inserted antegrade over the puncture wire 220 into the kidney with the radiopaque segment 206 providing visibility to the clinician. (Note the puncture wire 220 is shown schematically in FIGS. 25A-25C; it should be appreciated that the various puncture wires disclosed herein can be utilized). In FIG. 25B, the puncture wire 220 is removed and an endourology wire 230 is inserted through the proximal end into the lumen of catheter 200, emerging through the distal opening 208 of the catheter 200. FIG. 25C illustrates the catheter 200 advanced into the ureter and the endourology wire 230 removed. (Note the endourology wire 230 is shown schematically in FIGS. 25A-25C as it should be appreciated that the various endourology wires disclosed herein can be utilized) Note the endourology wire 230 can be removed through the proximal opening in the catheter 200 in a direction opposite its initial insertion through the lumen.

One example of the method of use of the catheter 200 will now be described, it being understood that catheter 210 can be used in the same fashion. The method steps in this example are as follows:
1. A flexible ureteroscope is positioned in a selected renal calyx (in the same manner as discussed above).
2. The puncture wire is passed through the working channel of the ureteroscope, through papilla, flank tissue, and outside of flank skin (in the same manner as discussed above).
3. A coaxial (or single lumen) exchange catheter with radiopaque tip or radiopaque filler (e.g., catheter 200 or catheter 210) is advanced over the emergent puncture wire (over the distal tip), in a direction opposite the puncture path, into the collecting system (see e.g., FIG. 25A).
4. Fluoroscopic imaging demonstrates the position of the tip of the exchange catheter.
5. When the tip of the exchange catheter is in the portion of the collecting system preferred by the surgeon (kidney or ureter), the puncture wire (and any inner coaxial catheter or inner dilator) is removed.
6. A new endourology wire is inserted into the proximal end of the exchange catheter (FIG. 25B), and advanced from outside to inside (in a direction opposite the original direction of puncture of the puncture wire), thus providing a precise positioning of the new endourology wire in relation to the urinary collecting system (kidney or ureter). For example, if the surgeon prefers the endourology wire to terminate in the kidney, this will be possible because the exchange catheter tip is radiopaque and the position of the open distal end (e.g., opening 208 of catheter 200 or opening 218 of catheter 210) is thus known in relation to the collecting system anatomy.

In one embodiment of this system, the nephrostomy puncture wire has various properties along its length as shown in FIG. 27. The puncture wire 250 has a thinner distal segment 254 proximal to sharp tip 258 and a thicker proximal segment 252 proximal of distal segment 254. The thicker segment 252 tapers at region 256 to the thinner segment 254. The thinner distal segment 254 proximal to sharp tip 258 is ideal for puncture through the flank while permitting endoscope deflection during puncture path targeting, and the thicker proximal segment 252 is stiffer to allow advancement of the exchange catheter system (single or coaxial catheter) with reduced risk of kinking of wire during this process. Ways to create a stiffer proximal segment 252 include for example precision grinding to comparatively reduce the diameter of the distal segment 254 or adding material to proximal wire segment 252, e.g., wire coiling overtop wire mandrel. Other methods to create the segments of different thicknesses are also contemplated. It should also be appreciated that the lengths of the thicker and thinner segments can vary from that shown in FIG. 27. Additionally, it is also envisioned that the puncture wire can have more than two thicknesses along a length.

A kit is contemplated containing a nephrostomy puncture wire with a tip-protective sheath and releasable clamp for securement of the wire and sheath, and an exchange catheter with the feature of a radiovisible tip (e.g., platinum band at the tip or doping of the partial or the entire exchange catheter itself so that tip is visible along with the portions of the catheter or the entire catheter).

### Dual Function Nephrostomy Puncture Wire

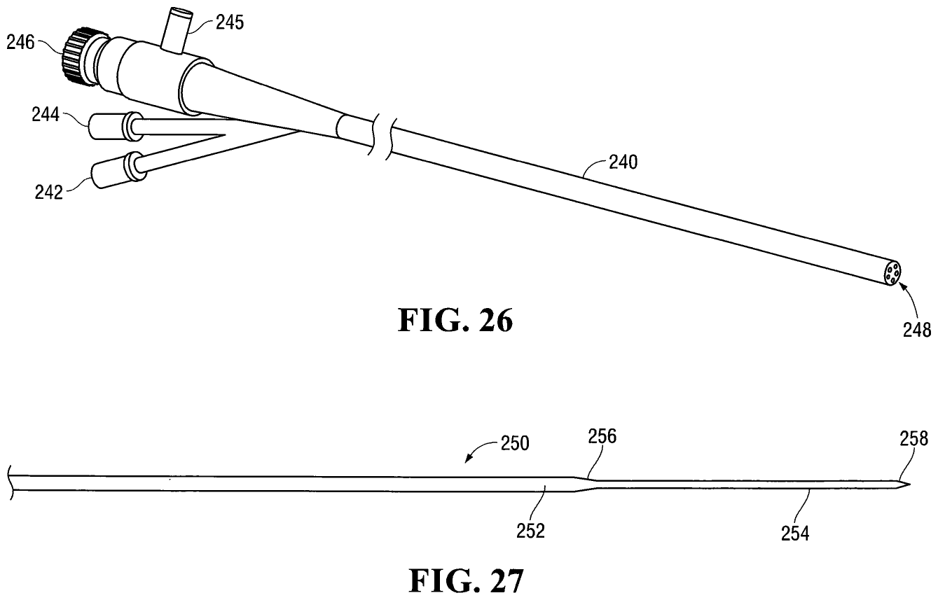

Nephrostomy tracts are generally created to permit placement directly into the kidney from the flank typically of endoscopes of various configurations to perform an endourologic procedure (typically renal stone removal, but also cancer treatment, reconstruction, stricture treatment, robotics, etc.). This class of endoscopes typically has an illumination function, a video function, a fluid channel for fluid flow, such as for irrigation, drainage, etc., and a working channel for instrumentation insertion (such as a laser, basket, etc.) FIG. 26 shows an example of such endoscope. designated generally by reference numeral 240 and having a lens 246 for visualization, port 245 for illumination, port 242 communicating with a fluid channel in the endoscope 240 and port 244 communicating with a working/instrument channel/lumen of the endoscope 240. Note that these channels sometimes serve dual functions e.g., one channel can be used for both irrigation and instrument insertion. Other instruments advanced over a wire into the kidney may include catheters with lumens to deliver medications, e.g., anticancer therapies, image enhancing pharmaceuticals for robotic surgery, etc.

The range of diameters of these endoscopes (or catheters) are generally between 8 French and 27 French. These mini-perc and micro-perc sets for minimally invasive percutaneous techniques can be between 10 French and 24 French to allow the physician to perform a percutaneous endourology procedure through smaller diameter tracts than the historically popular 30 French tracts. The mini-perc and microperc sets reduce the size of the skin incision, fascial defect, and renal defect to reduce trauma to the tissues while allowing effective endourologic surgery to be performed.

In the setting of retrograde nephrostomy creation for endourologic surgery. the retrograde nephrostomy wire is typically deployed through a flexible ureteroscope positioned in a renal infundibulum, with the ureteroscope tip facing the renal papilla. The puncture wire travels straight, through the renal capsule, perinephric fat, and flank musculature.

The present invention provides in some embodiments a retrograde nephrostomy puncture wire with two functions: 1) retrograde nephrostomy puncture and 2) bridge over which endourology scopes or sheaths, with tapered dilating tips, are advanced into the kidney. This dual function nephrostomy puncture wire eliminates the need to exchange the puncture wire for an endourology working wire, and in some applications may permit elimination of the need for a wire exchange catheter e.g., coaxial or single lumen wire exchange catheter systems.

The dual function nephrostomy puncture wire can be of single diameter and stiffness along its length or may have different properties along its length. FIG. 27, as described above, provides an example of a puncture wire having different properties along its length with its distalmost tip 258 sharpened for tissue puncture and the segment 254 proximal to the distal tip 258 of a diameter to provide enough flexion ability of the positioning endoscope (typically a flexible ureteroscope) while having enough stiffness to puncture through tissue. The segment 250 proximal to the segment 254 will be stiff enough, due to its thickened (larger diameter) portion, to allow advancement of the endourology scopes (e.g., in mini-pert or micro-perc techniques) with tapered/dilator tip or catheter to pass over this puncture wire 258 from outside the flank to inside the kidney in an antegrade direction, i.e., a direction opposite the initial retrograde renal puncture. The proximalmost segment 250 that serves as a bridge over which the endourology scope (e.g., in mini-perc or micro-perc techniques) with tapered/dilator tip (or catheter) passes may have a property to prevent kinking. For example, the proximal segment 250 may have a larger diameter with increased stiffness compared to the narrower segment 254 just proximal to the puncture point 258. Puncture tip 258 will have less stiffness than the proximal segment 250. This increased stiffness of the proximal segment 250 may be achieved with a larger diameter, or a secondary material over the wire mandrel, e.g., coil, as discussed above.

Described below are several examples of methods of use of the dual puncture wire. Note the puncture wire 220 shown in these dual function puncture wire examples has a single diameter along its length except for the distal pointed/sharp tip. However, it should be appreciated that other puncture wires, such as puncture wire 250 having varying stiffnesses along its length, can be utilized. Therefore, the methods described in the examples below are fully applicable to use of puncture wire 250 as well as fully applicable to other puncture wires having other dimensions, properties, penetrating tips, etc. Note the ureteroscope shown in the dual function puncture wire examples is the ureteroscope 40 of FIG. 3 described above. However, it should be appreciated that other ureteroscopes such as ureteroscope 240 of FIG. 26 can be utilized. Therefore, the methods described in the examples below are fully applicable to use of ureteroscope 240 as well as other scopes having other ports, channels, illumination, video, etc.

### Example 1 of Dual Function Wire:

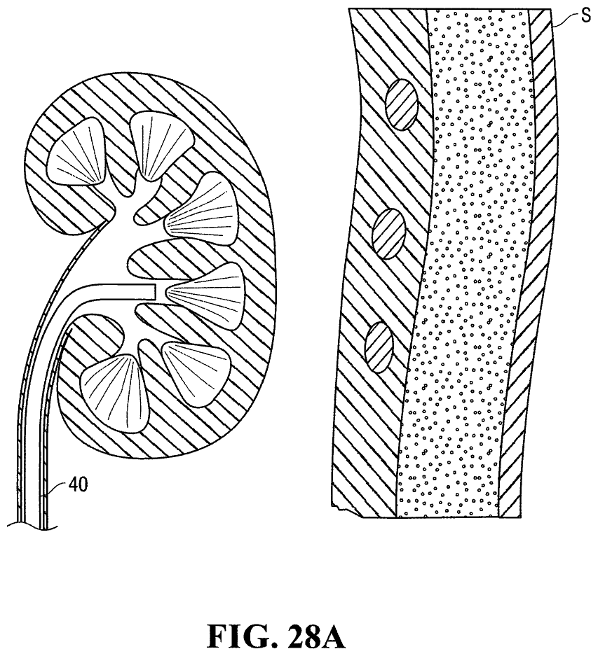

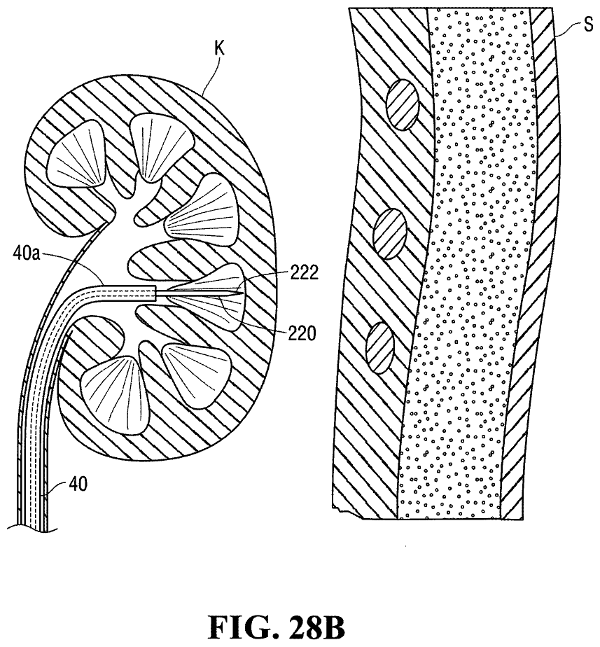

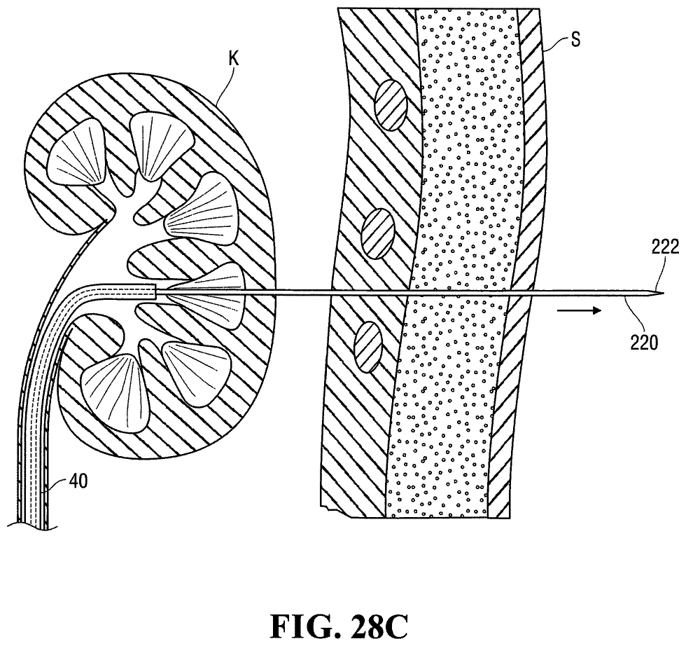

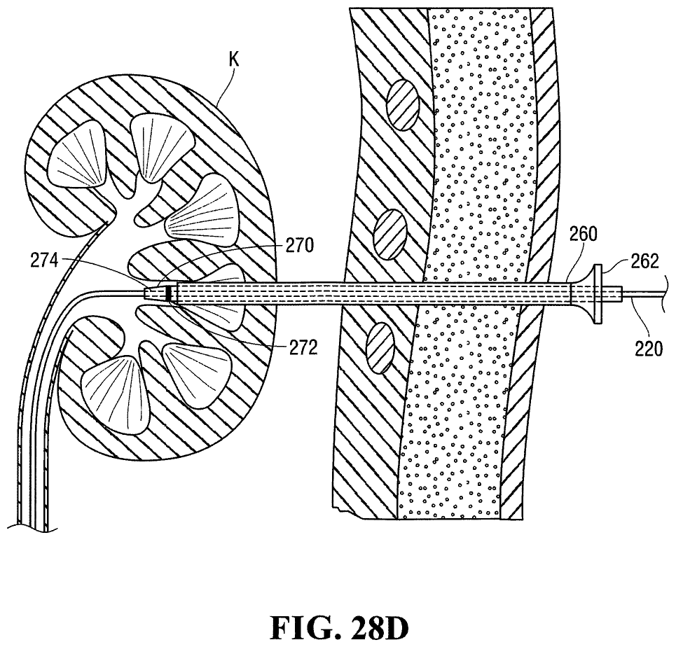

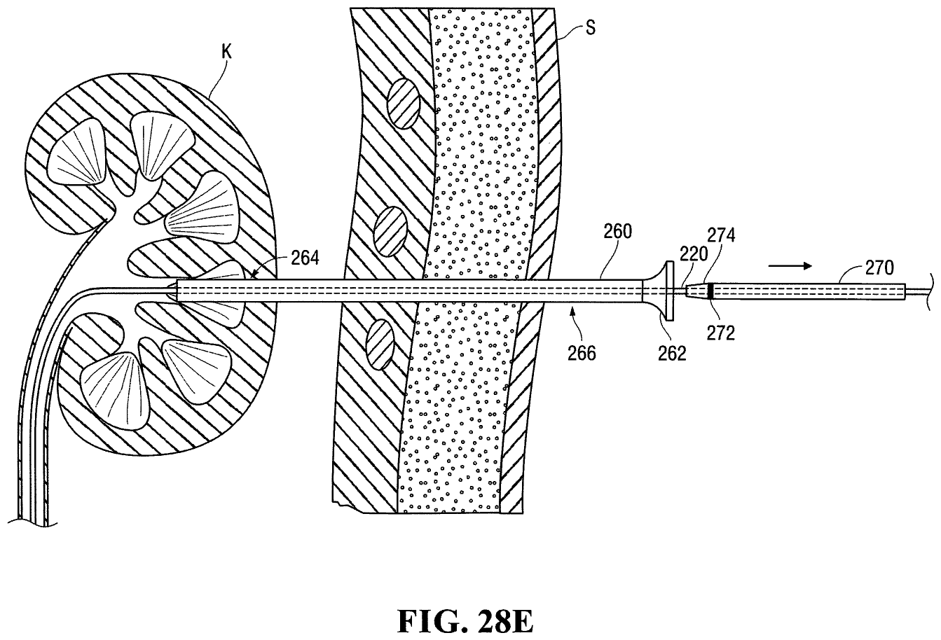

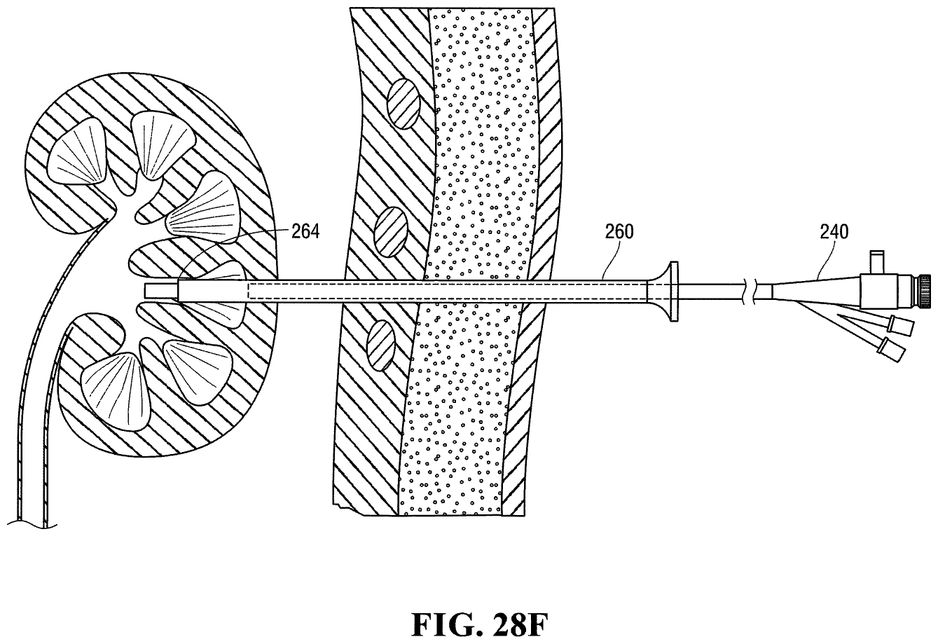

The steps of this procedure are as follows and are shown in FIGS. 28A-28F:
1. Position the flexible ureteroscope 40 in a physician selected renal calyx for puncture (FIG. 28A).
2. Insert the puncture wire 220 into the working channel of the flexible ureteroscope 40 (or ureteroscope 24 or other scope), until the puncture end/tip 222 end is seen emergent from the end distal 40a of the ureteroscope (FIG. 28B).
3. Release the pin vise lock to release the puncture wire 220 in relation to the sheath, and advance the puncture wire 220 relative to the sheath through the renal papilla, flank tissue, and skin S (FIG. 28C).
4. Advance the duo of an outer sheath 260 (FIG. 28D) and removable inner dilator 270 with tapered tip 274 over the emergent nephrostomy puncture wire 220 in a direction opposite nephrostomy puncture of the puncture wire 220 until it enters the kidney K. (Note as shown in FIG. 28D, the dilator 270 is positioned within a lumen of the outer sheath 260, and extends proximally of hub 262 of dilator 260, and can include a radiopaque marker, e.g., band or filler, as in catheters 202, 210 described above).
5. Remove the inner dilator 270 in the direction opposite the direction of initial insertion over the puncture wire 220 (FIG. 28E), leaving the distal end 264 of sheath 260 in the kidney and the proximal end 266 and hub 262 out of the flank.
6. Insert endoscope 240 as shown in FIG. 28F (having the illumination, visualization, marking channel as described herein) through the sheath 260 to enable the endourology procedure to be performed.

### Example 2 of Dual Function Wire:

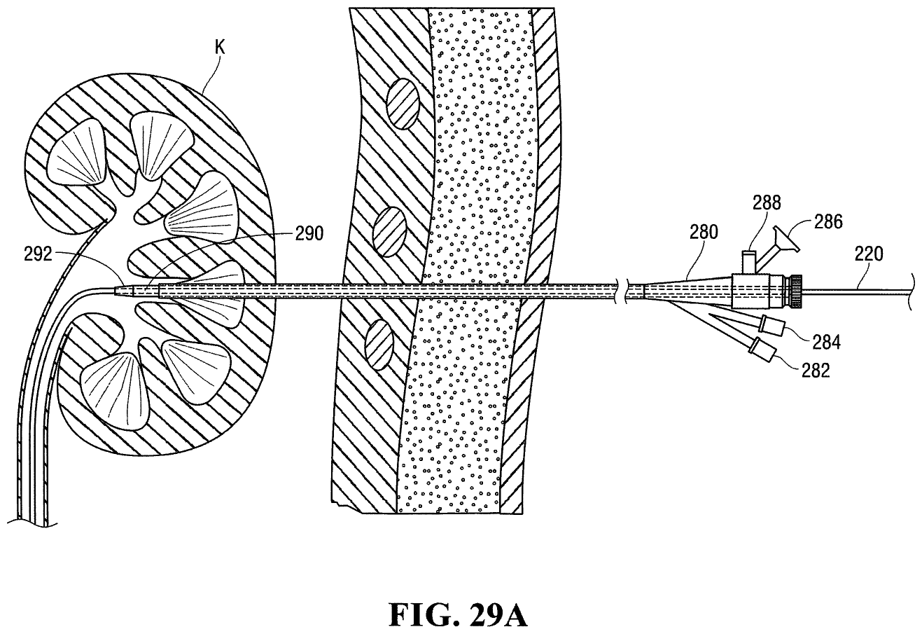

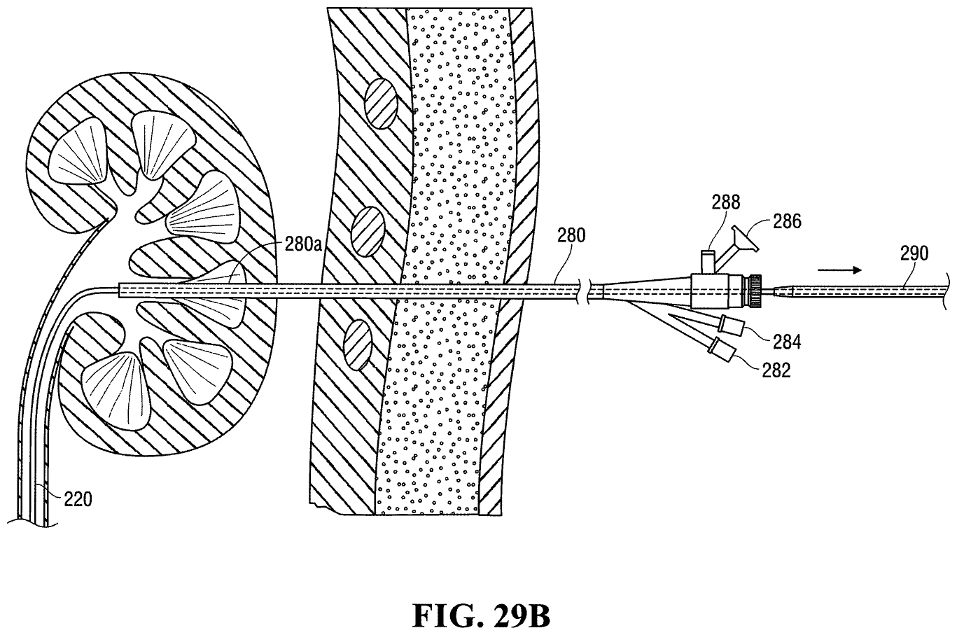

The steps of this procedure are as follows and are shown in FIGS. 29A-29B. Note steps 1-3 are the same as in Example 1; the difference being in steps 4 and 5:
1. Position the flexible ureteroscope 40 in a physician selected renal calyx for puncture (FIG. 28A).
2. Insert the puncture wire 220 into the working channel of the flexible ureteroscope 40 (or ureteroscope 24 or other scope), until the puncture end/tip 222 end is seen emergent from the distal end 40a of the ureteroscope (FIG. 28B).
3. Release the pin vise lock to release the puncture wire 220 in relation to the sheath, and advance the puncture wire 220 through the renal papilla, flank tissue, and skin (FIG. 28C).
4. Advance a nephroscope 280 (FIG. 29A) with removable inner dilator 290 with tapered tip 292 over the emergent puncture wire 220 in a direction opposite the advancement of nephrostomy puncture of the puncture wire 220.
5. Remove the inner dilator (FIG. 29B) in a direction opposite the direction of initial insertion over the puncture wire 220 leaving the distal end 280a of the nephroscope 280 in the kidney and the proximal end 280b extending out of the flank. Note nephroscope 280 has port 288 for light/illumination, port 286 for imaging, port 282 for irrigation/drainage and port 286 communicating with a working channel of the scope 280 for insertion of instrumentation to perform the procedure.

### Example 3 of Dual Function Wire:

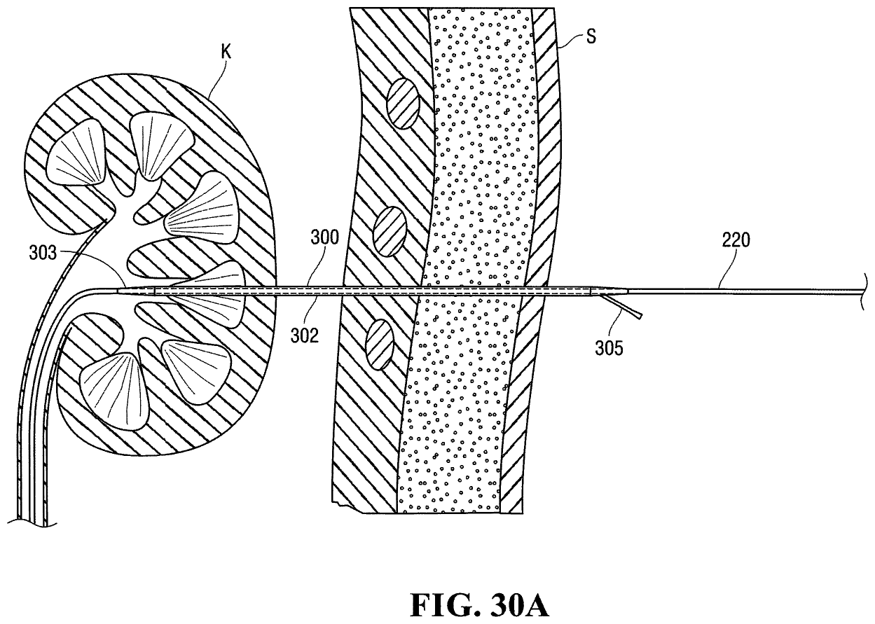

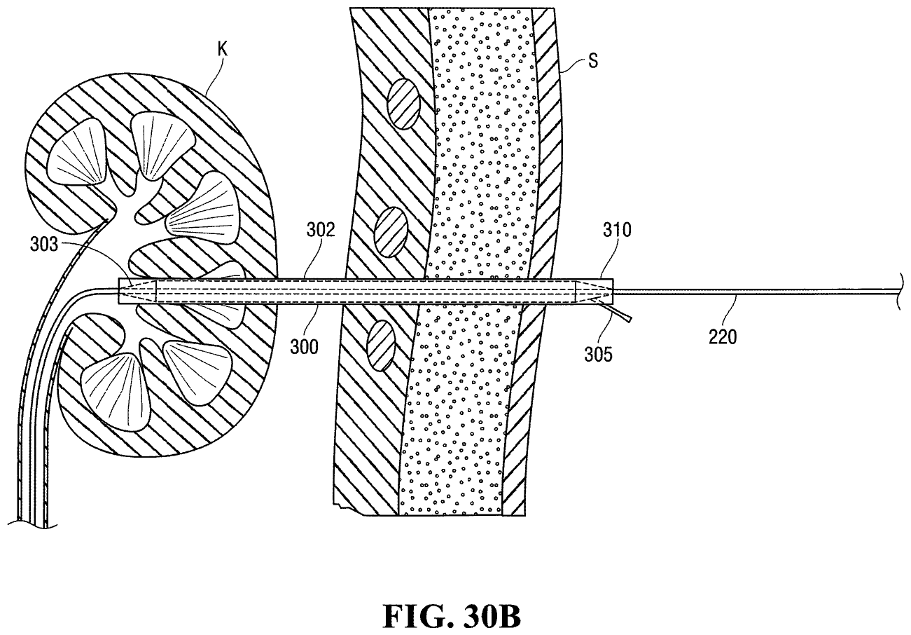

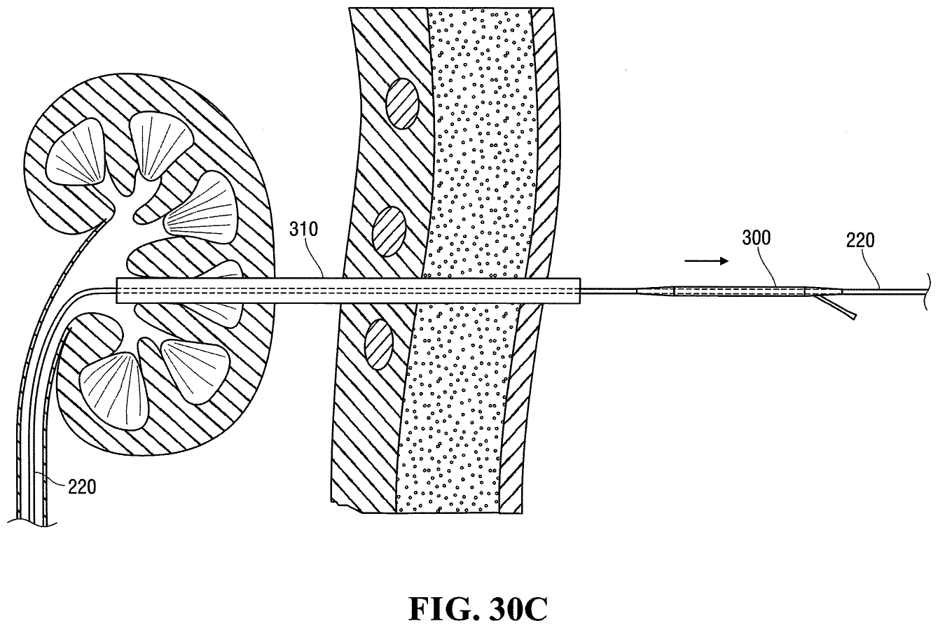

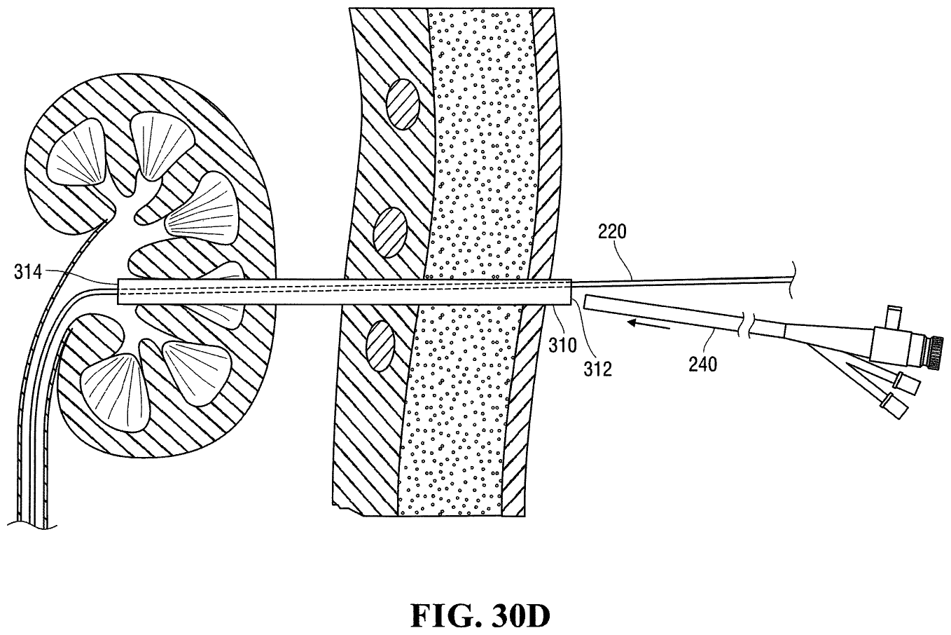

The steps of this procedure are as follows and are shown in FIGS. 30A-30D. Note steps 1-3 are the same as in Example 1; the difference being in steps 4-7.
1. Position the flexible ureteroscope 40 in a physician selected renal calyx for puncture (FIG. 28A).
2. Insert the puncture wire 220 into the working channel of the flexible ureteroscope 40 (or ureteroscope 24 or other scope), until the puncture end/tip 222 end is seen emergent from the distal end 40a of the ureteroscope (FIG. 28B).
3. Release the pin vise lock to release the puncture wire 220 in relation to the sheath, and advance the puncture wire 220 through the renal papilla, flank tissue, and skin (FIG. 28C).
4. Advance a balloon dilation catheter 300 (FIG. 30A) over the emergent puncture wire 220 in a direction opposite the direction of nephrostomy puncture by puncture wire 220 until the tip 303 of the balloon 302 is in the kidney.
5. Inflate the balloon 300 via injection of fluid through port 305 into a lumen in the catheter communicating with an interior of the balloon 300 and advance a nephrostomy sheath 310 (FIG. 30B) in the same direction as insertion of the balloon catheter 300 over the balloon 302 into the kidney.
6. Deflate and remove the balloon catheter 300, leaving the outer sheath 310 (in place (FIG. 30C).
7. Insert endoscope 240 (FIG. 30D) in a direction opposite the direction of puncture wire 220 insertion, through a proximal opening 312 in outer sheath 310 and through a lumen in the outer sheath 310 and into the kidney to enable the endourology procedure to be performed. Note the sheath 310 can have one lumen for the puncture wire 220 and a second lumen, independent of the first lumen, for the scope 240 or alternatively they can both be positioned in the same lumen in the sheath 310.

### Example 4 of Dual Function Wire:

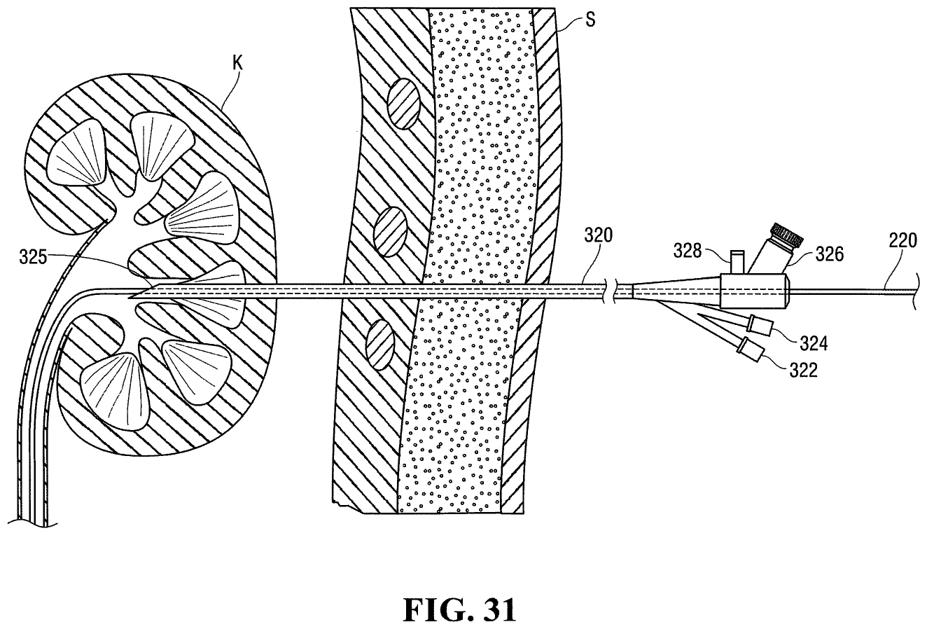

The steps of this procedure are as follows and are shown in FIG. 31. Note steps 1-3 are the same as in Example 1; the difference being in step 4.
1. Position the flexible ureteroscope 40 in a physician selected renal calyx for puncture (FIG. 28A).
2. Insert the puncture wire 220 into the working channel of the flexible ureteroscope 40 (or ureteroscope 24 or other scope), until the puncture end/tip 222 end is seen emergent from the distal end 40a of the ureteroscope (FIG. 28B).
3. Release the pin vise lock to release the puncture wire 220 in relation to the sheath, and advance the puncture wire 220 through the renal papilla, flank tissue, and skin (FIG. 28C).
4. Insert endoscope 320 (FIG. 31) with tapered tip 325 over the emergent nephrostomy puncture wire 220 in a direction opposite nephrostomy puncture into the kidney to enable the endourology procedure to be performed. Note endoscope 320 has port 328 for light/illumination, port 326 for imaging, port 322 for irrigation/drainage and port 324 communicating with a working channel of the scope 320 for insertion of instrumentation to perform the procedure.

### Example 5 of Dual Function Wire:

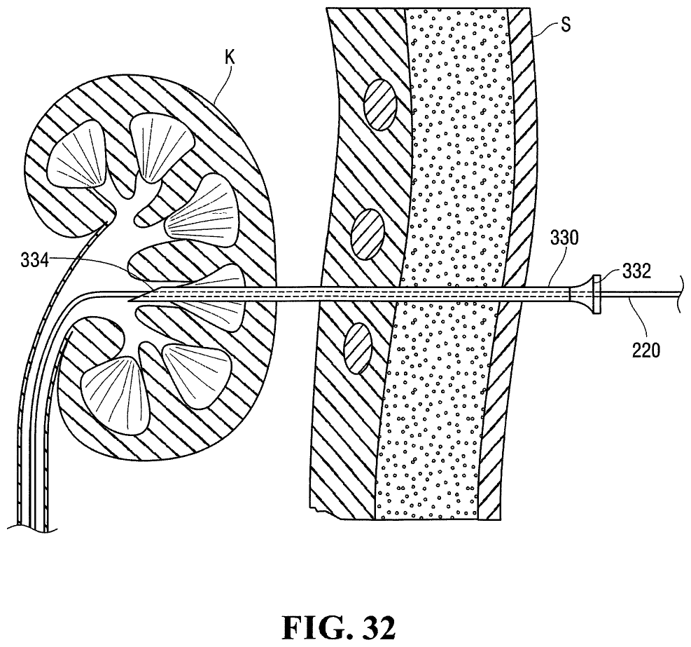

The steps of this procedure are as follows and are shown in FIG. 32. Note steps 1-3 are the same as in Example 1; the difference being in step 4:
1. Position the flexible ureteroscope 40 in a physician selected renal calyx for puncture (FIG. 28A).
2. Insert the puncture wire 220 into the working channel of the flexible ureteroscope 40 (or ureteroscope 24 or another scope), until the puncture end/tip 222 end is seen emergent from the distal end 40a of the ureteroscope (FIG. 28B).
3. Release the pin vise lock to release the puncture wire 220 in relation to the sheath, and advance the puncture wire 220 through the renal papilla, flank tissue, and skin (FIG. 28C).
4. Insert catheter 330 (FIG. 32) with tapered tip 334 over the emergent nephrostomy puncture wire 220 in a direction opposite nephrostomy puncture into the kidney to enable the endourology procedure to be performed, e.g., permit planned endourology diagnosis or therapy. The catheter 320 can have multiple lumens and may have a string or other mechanism attached to a distal region to effect a curl via a proximal pulling motion to secure the catheter in the kidney. Instrumentation can be inserted through the catheter 330 to perform the procedure.

### Puncture Wire Deviation

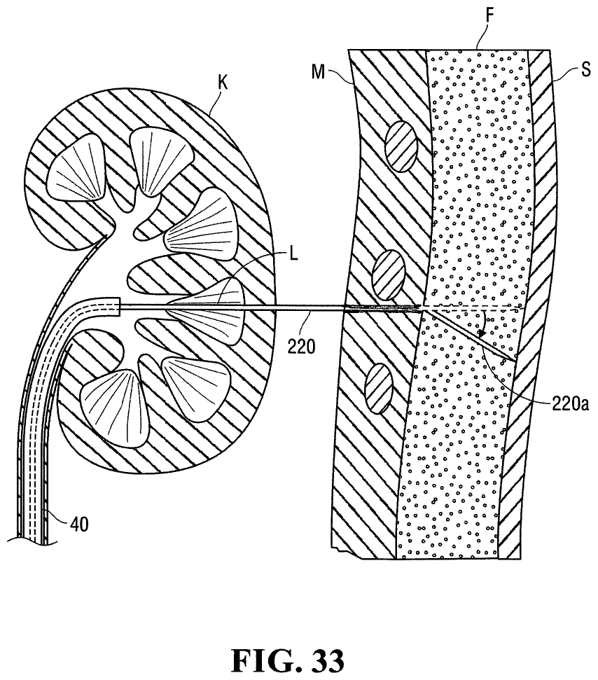

During the initial retrograde nephrostomy puncture wire advancement, the puncture wire passed through the flank fascia, traverses the flank fat and emerges at the flank skin. The subcutaneous fat has a variable thickness depending on the patient's body habitus. The retrograde nephrostomy puncture wire can deflect slightly while traversing this subcutaneous fat, and thus may deviate from its precise alignment with the infundibular long axis (see axis L of FIG. 33). Often such slight deviation includes a more caudad path for the puncture wire such as shown in FIG. 33. In FIG. 33 the normal path is shown in phantom and the deviated path within the fat F (between the flank musculature/fascia M and skin S) is shown in solid line wherein the puncture wire 220 would emerge through the skin S out of alignment). In patients with longer puncture wire paths through the subcutaneous fat, more deviation from the initial wire path can be seen as a simple extrapolation of the initial slight wire path deviation. As such, the final stage of emergence of the puncture wire through the skin can be slightly difficult, again commonly with obese patients.

Fluoroscopy is a guide to the path of the puncture wire advancing through the subcutaneous fat. Two limitations of the current system of retrograde nephrostomy are: 1) Fluoroscopy is a two-dimensional imaging modality; the wire path is three dimensional, so exact position of the wire in three dimensions is not knowable using biplanar fluoroscopy, unless more advanced fluoroscope rotations are employed. These are difficult to learn and teach and use ionizing radiation; and 2) There is not currently a retrieval system for a wire in the subcutaneous fat; wire emergence at the flank is passive at this time.

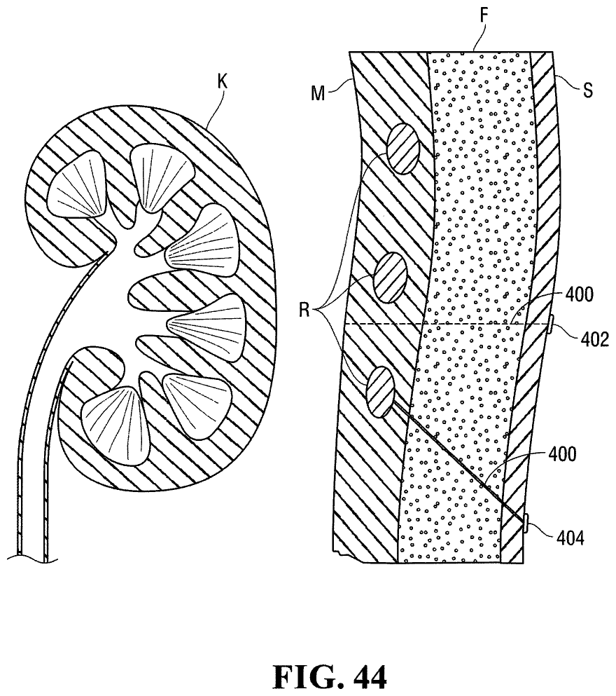

That is, in some cases, such as in obese patients with longer paths to the flank skin, the puncture wire advancement does not emerge from the flank skin before it deflects from its original course or stalls in subcutaneous or flank tissues. Despite the wire not emerging at the skin, the complex anatomy of the kidney and flank muscles have been successfully and safely penetrated; in these cases, the only remaining task is to deliver the wire from the subcutaneous tissues, or flank tissues, to complete the puncture portion of the procedure, prior to applying catheter(s) over the emergent wire for wire exchange (or prior to applying endoscopes over the emergent wire as described herein). The present invention provides several strategies to effect retrieval of the puncture wire from the subcutaneous tissues. These may include, but are not limited to: (1) ultrasound localization of the wire, and then incision of the skin and retrieval of the puncture wire tip with a clamping device; (2) fluoroscopy based localization of the wire, and then incision of the skin and retrieval of the puncture wire tip with a clamping device or; (3) preplacement of radiopaque or ultrasound visible markers on the flank skin, to inform the positioning of the ureteroscope tip prior to puncture wire advancement, or to inform a skin incision at the flank to retrieve an already deployed puncture wire. For example, if one or more radiopaque markers are placed on the skin, they can serve as a reference point to the fluoroscopic position of the puncture wire in the subcutaneous tissues to direct a skin incision to find and capture the puncture wire tip that has not emerged from the skin. This is shown for example in FIG. 44. FIG. 44 illustrates use of a fluoroscopically visible skin marker 402 that rests on the skin or a fluoroscopically visible rib marker 404 or a needle like marker that contacts and secures releasably to the rib R itself in the muscle region M, to enable guidance for retrograde nephrostomy puncture by increasing knowledge of the position of the rib during puncture.

Regardless of which method to complete wire retrieval from the flank is employed, subsequent wire exchange at the flank may proceed with either coaxial catheter, single lumen catheter, or serial dilators to exchange the puncture wire for a larger diameter endourology working wire or sheaths, catheters or scopes can be placed over the emergent puncture wire in accordance with some of the methods described herein.

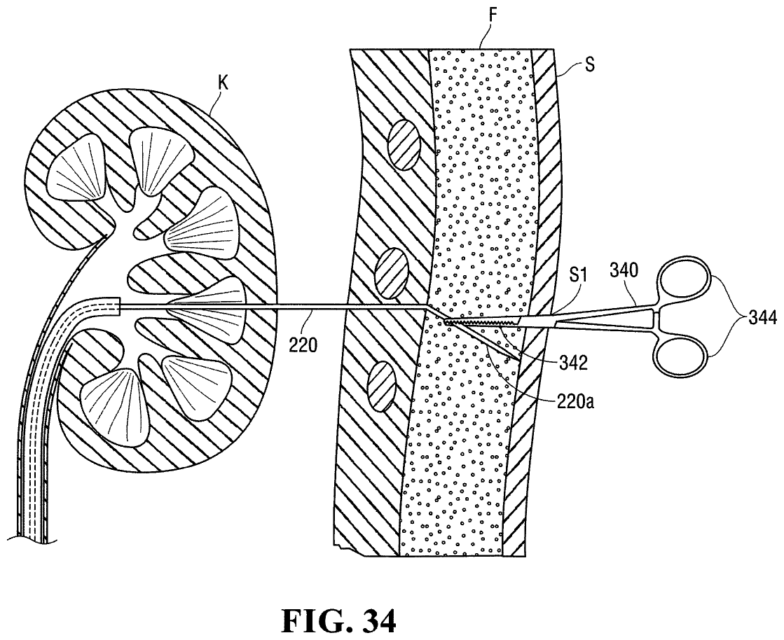

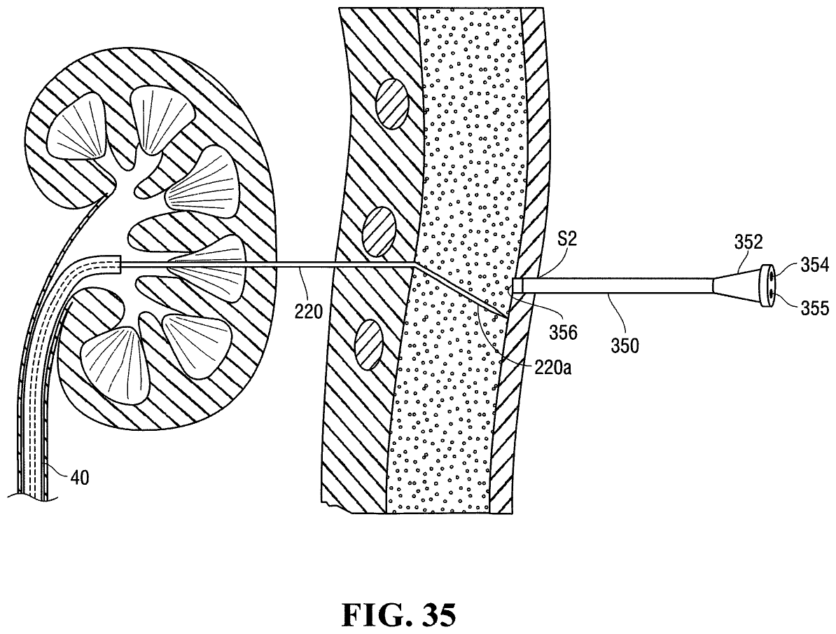

FIGS. 34 and 35 illustrate methods of the present invention in accordance with some embodiments to manage the puncture wire path deviation. In one approach/method to manage this deviation problem, a clamping member 340 (FIG. 34) is advanced through a separately made flank skin incision S1 and fluoroscopy is employed to direct the clamping member 340 through the skin S toward the puncture wire 220 in the subcutaneous fat F. The puncture wire tip 220a or other portions of the body of the wire 220 as shown in FIG. 34 can be captured by the jaws 342 of clamping member 340, the jaws 342 (with grasping surfaces, e.g., teeth or serrations) opened and closed by movement of the handle loops 344 in a scissors-like fashion. Note clamping member 340 is an example of one type of clamping member that can be utilized, it being understood that other clamping members are also envisioned for grasping and moving the puncture wire.

To the extent that each fluoroscopy image is two dimensional, for any one fluoroscopy image, the wire's position in the third dimension remains unknown. Typically, this unknown dimension is the ‘anterior/posterior’ position, though this can be any third dimension. Thus, in this approach of the present invention, the surgeon may need to attempt wire capture at more than one position in the third, unknown plane, until the wire is captured. Alternatively, the fluoroscope can be rotated to inform the third plane. Once the wire 220 is captured, the clamp 340 is drawn out of the skin thereby completing the retrograde nephrostomy procedure as the captured wire 220 can be advanced out the flank. The puncture wire 220 can then be exchanged with a flexible catheter advanced over the puncture wire 220, where this catheter will enter traverse the flank tissues, kidney, and ureter, to permit “through and through” renal access. This exchange catheter can be a single lumen, dual lumen, or coaxial in configuration as described in detail herein. Alternatively, the puncture wire could be a dual function wire as described above and a sheath, catheter or scope passed over the emergent puncture wire to enable the endourology procedure.

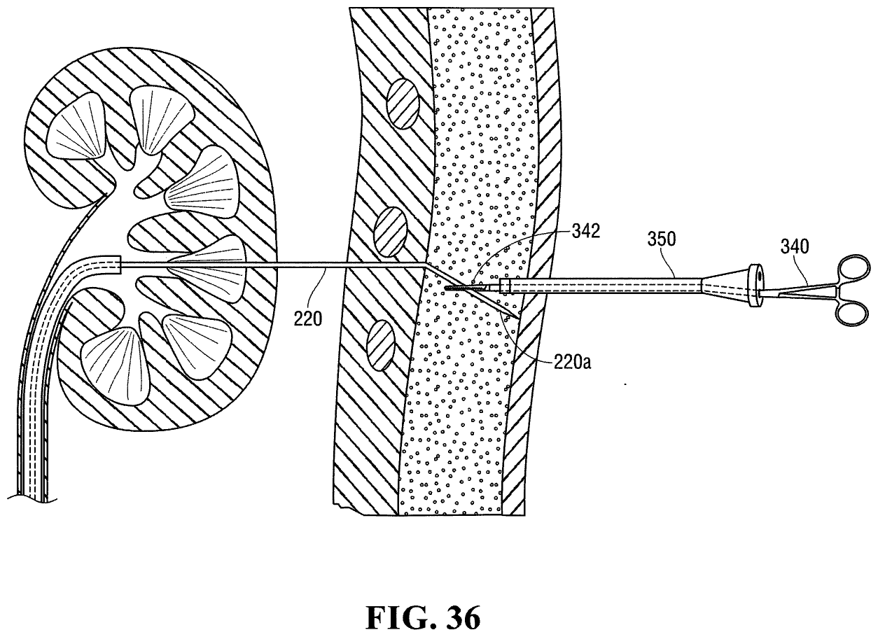

In another approach/method of the present invention for wire alignment, a multi-function port is utilized to provide access for a clamping instrument rather than direct insertion as in the embodiment of FIG. 34. Multifunction port 350, as shown in FIG. 35, has two proximal access openings to channels 354, 355 at hub 352 communicating with lumens extending through the port 350 and open at distal end 356. Port 350 is inserted in a separately made flank skin incision S2 (see FIG. 35) which can be the same site or different than S1 of FIG. 34 showing the direct clamp insertion method. With the aid of this port 350, the puncture wire 220 is retrieved via a clamping member 340 inserted through a channel in the port (FIG. 36). The clamping member 340 can be the same as utilized in the method of FIG. 34 or a different clamping member can be utilized.

The port 350 includes a light to illuminate the subcutaneous or perinephric space (though the subcutaneous space is considered preferable), compatibility with, or a built in camera which connects to a display monitor for visualization, a channel/lumen 354 for fluid or insufflation and drainage, and a working channel 355 to permit advancement of a grasping (clamping) member to capture the puncture wire 220 seen under direct endoscopic vision. In alternate embodiments, the fluid channel 354 could also be used as the working channel for the grasper so fewer channels could be provided. An integrated grasper function is also contemplated wherein for example the grasper member is part of and not removable form the port. Preferably, the port includes an access port and channel (see e.g., channel 354) capable of transmitting a fluid or gaseous medium to create tissue separation to permit increased visibility of the puncture wire in the subcutaneous or perinephric space though the subcutaneous space. Once the port 350 is in position, the tissue working space is increased with fluid or gas, the light/camera (illumination/imaging) system is employed directed toward the puncture wire 220, and the grasper 340 is used to capture the puncture wire 220 either at its tip or along its length. Note the grasper of the embodiments of FIGS. 34 and 36 can be utilized for grasping and moving puncture wires other than puncture wire 220 shown in the drawings.

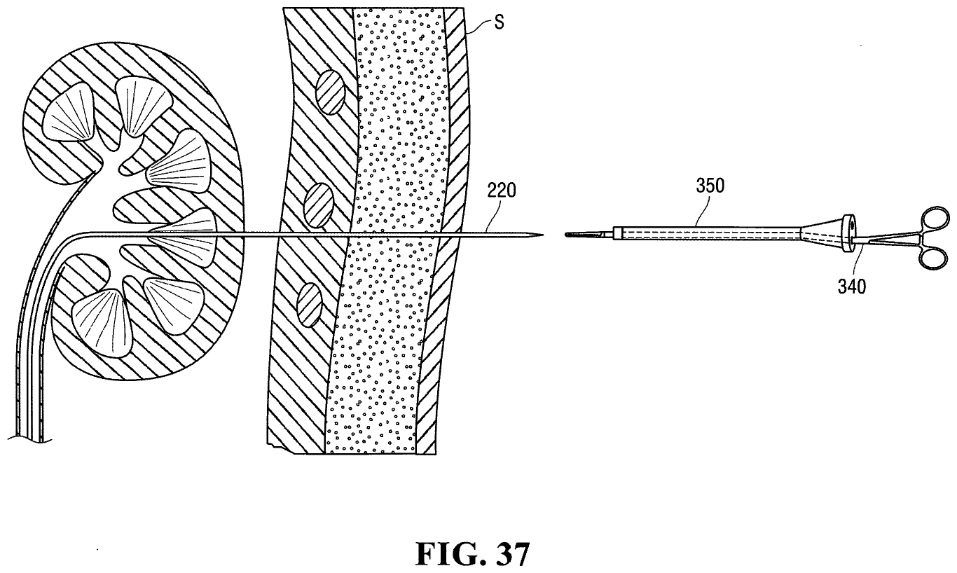

Note FIG. 37 illustrates the puncture wire 220 extending back to a straight path as it is retrieved by the clamping member; however, it should be understood that the clamping member can pull the puncture wire 220 along the deviated path through the skin S and flexible instruments can be advanced over the angled puncture wire 220 in the same manner as advancement of the straight wire. Also note that the layer of Muscle M, Fat F and skin S are shown schematically and the thicknesses of the layers can vary from that shown in the drawings.

Once the wire 220 is securely grasped by the clamping member 340, two approaches/methods are contemplated, referred to herein as Approach (method) A and Approach (method) B for convenience.

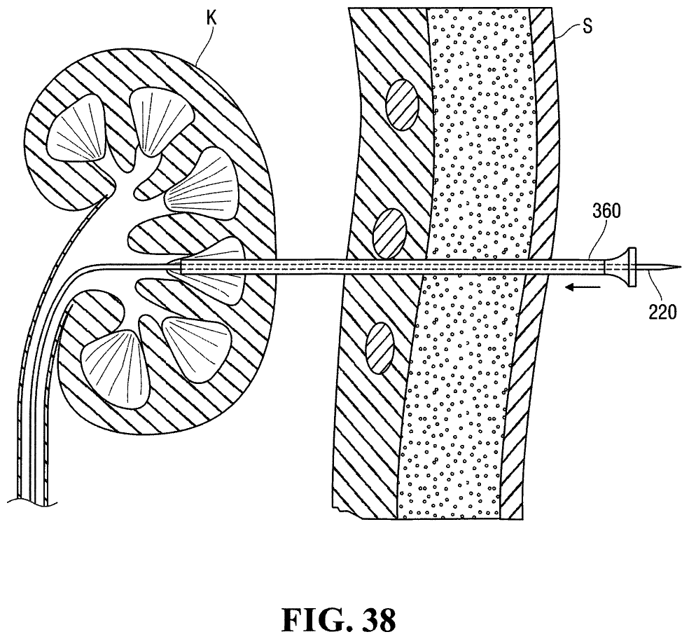

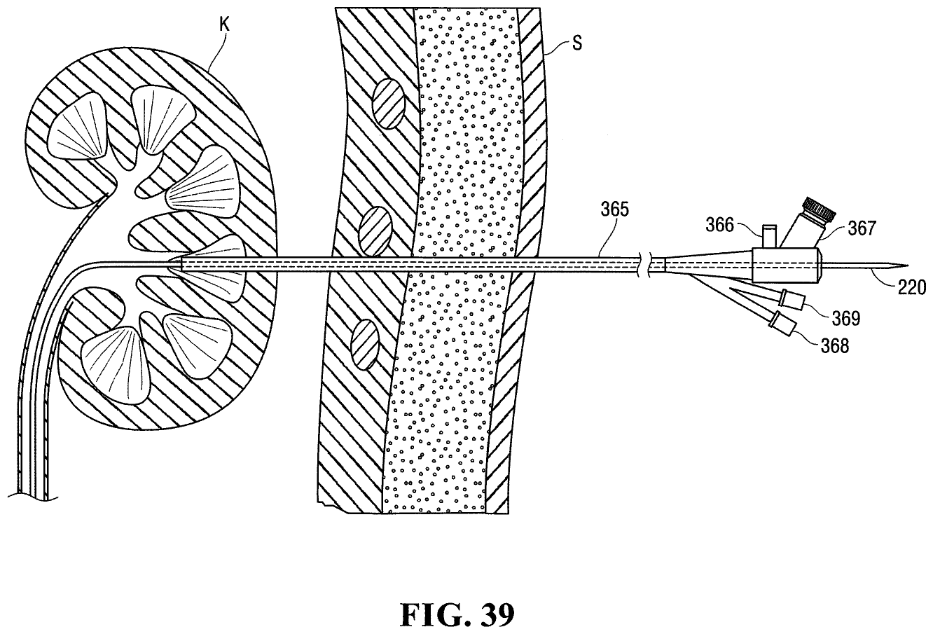

In Approach A, the port 350 and captured wire 220 are removed through the skin S in the same direction as the retrograde puncture advancement, thereby completing the retrograde nephrostomy creation procedure. The clamping member 340 is released from the puncture wire 220 and removed with the port 350 (FIG. 37). The emergent puncture wire 220 which has been retrieved and released from the clamping member 340 can then be either a) exchanged with a flexible exchange catheter (coaxial or single lumen) advanced over the puncture wire 220 in a direction opposite to puncture wire advancement (see FIG. 38), wherein this catheter 360 will enter traverse the flank tissues and into the kidney (and possibly ureter to permit “through and through” renal access), or (2) an endourology scope or catheter or sheath can be advanced directly over the emergent nephrostomy puncture wire 220 into the kidney (as in the dual function nephrostomy puncture wires described herein where they provide renal/flank puncture and a bridge to load a renal endoscope). FIG. 39 illustrates an endoscope 365 rather than an exchange catheter inserted over the puncture wire. Endoscope 365 has illumination 366, imaging 367, port 368 for fluid insertion and/or drainage and port 369 for instrument insertion into the working channel.

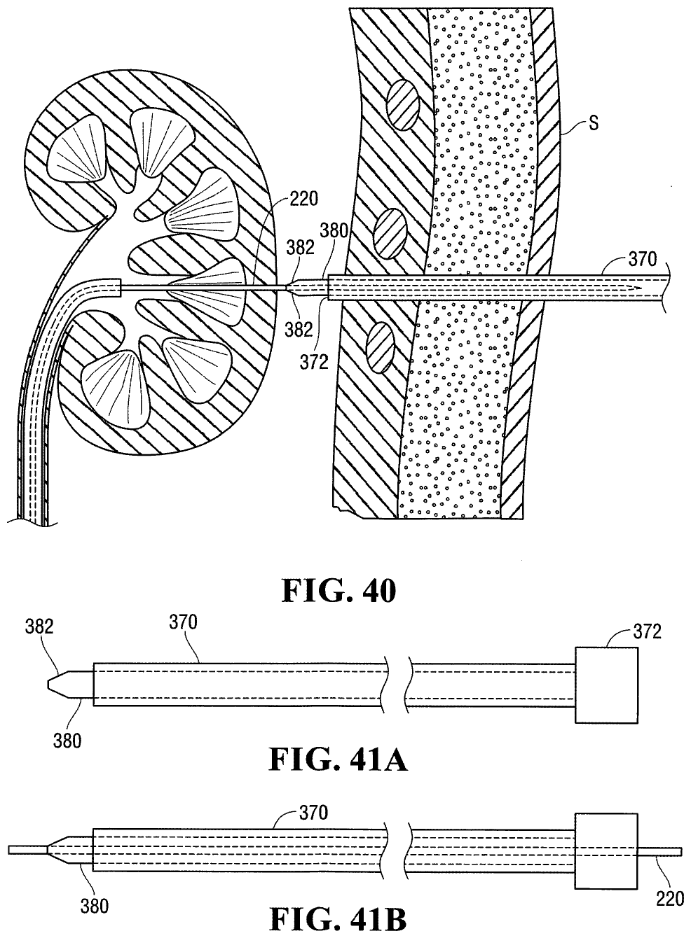

In Approach B (FIGS. 40-43), the renal endoscope (e.g., miniperc or microperc) also contains a puncture wire retrieval system and is advanced into the kidney for the planned endourology procedure, e.g., kidney stone breakage/removal, tumor treatment, etc. after wire capture. In approach B, after capture of the puncture wire 220, the port 370 with a tip configuration e.g., a tapered tip, to permit atraumatic tissue dilation to traverse the flank musculature and renal tissue (FIG. 40) is advanced into the kidney.

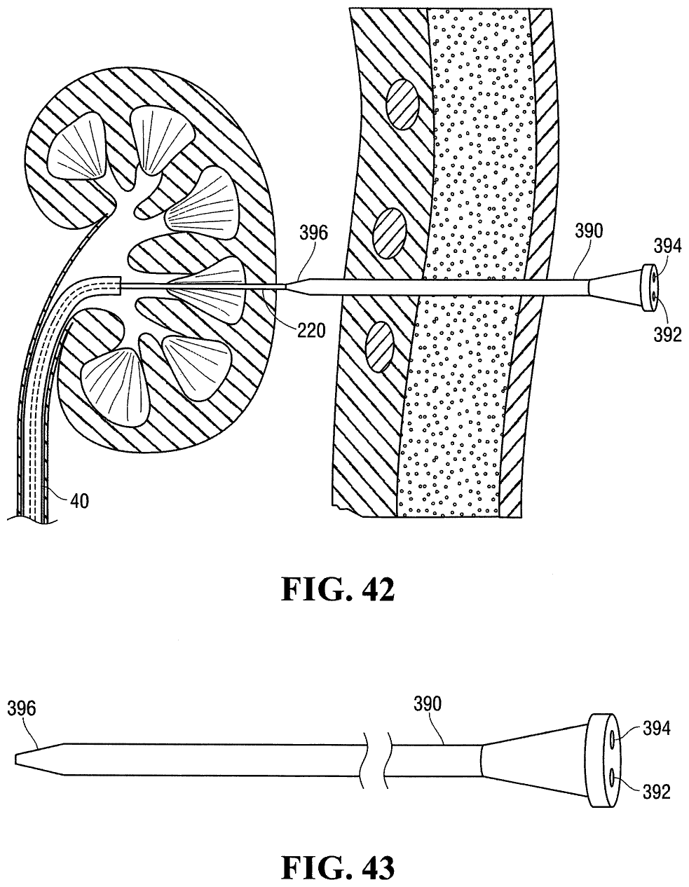

This atraumatic tip for tissue dilation may either be at the distal end of an intubating obturator 380 within a working channel of the port 370 and removable therefrom as shown in FIG. 40-41B, or alternatively part of the wire retrieval port itself so it forms a distal region of the port as in port 390 (with channels 392, 394) of FIGS. 42-43 having a dilating tip 396 extending distally therefrom. One of the channels 392, 394 of port 390 can be used to receive the puncture wire 220 for advancement of the port 390 over the wire 220; the other channel can be used for fluid insertion, drainage, instrument insertion, etc. In the embodiment where there is a separate (independent) dilating tip obturator as in FIG. 41A, once the puncture wire 220 is captured, the wire 220 is delivered through the distal end of the port and out the back (proximal opening) of the port 370 as shown in FIG. 41B. Over this wire 220, the obturator 380 is advanced until the atraumatic dilating tip configuration is created at the distal end of the port 370 as the obturator dilating tip 382 protrudes distally past the distal end of the port 370. Using the puncture wire 220 as a guide, the port 370 is advanced over the puncture wire 220 into the kidney, optionally employing direct vision endoscopy during the port advancement (optionally also fluoroscopy) until the port 370 is inside the renal collecting system. At this time, the dilating tip obturator 380 can be removed from the port 370 to permit advancement of laser/lithotripsy/basket/biopsy/endoscopes (flexible or rigid) or other diagnostic or therapeutic interventions through port 370. The same video endoscopy capability used for wire capture can be used for the planned endourology procedure in the renal collecting system, or alternatively a second/separate endoscopy capability could be used.

In an alternate embodiment, there are two separate ports; the first port to retrieve the puncture wire and the second port to effect the planned endourology procedure.

It should be appreciated that this concept of retrograde nephrostomy puncture wire directed targeting of renal access could be applied to treatment of renal tumors or other renal parenchymal diagnostics or therapeutics (beyond the collecting system space), wherein the retrograde puncture wire path, once controlled, is leveraged toward a directed and safe advancement of a diagnostic or therapeutic member (e.g., endoscope or port or catheter) over the controlled puncture wire in a direction opposite the retrograde puncture.

### Exchange Catheter

In the setting where an exchange catheter is used to change the emergent retrograde nephrostomy puncture wire at the flank, the method steps are as follows. Any of the puncture wires disclosed herein can be utilized with this method.
1. Position a flexible ureteroscope in a selected calyx.
2. Advance the nephrostomy puncture wire apparatus (puncture wire and sheath) through the working channel of the ureteroscope until the puncture tip of the puncture wire emerges from the ureteroscope.
3. Advance the puncture wire until it is emergent outside of the flank.
4. Advance over the emergent puncture wire an exchange catheter, which is advanced over the wire into the renal collecting system.
5. Remove the puncture wire and/or inner dilator (if the exchange catheter is a coaxial system).
6. Advance a new endourology wire into the exchange catheter (outer catheter if inner dilator portion of coaxial system was removed).
7. Remove the outer coaxial catheter.

In this setting, the subsequent endourology procedure may require tract dilation using a sheath and inner tapered dilator, a sheath/nephrostomy dilation balloon, etc., or a diagnostic/therapeutic catheter, e.g., chemotherapy catheter, nephrostomy catheter.

A kit is contemplated herein which would include the nephrostomy puncture wire and sheath, with releasable lock, an exchange catheter (single or coaxial lumen), an endourology working wire, and any of 1) a sheath/releasable inner dilator; 2) sheath/nephrostomy balloon; 3) (1) or (2) above AND either (a) a diagnostic/therapeutic catheter, or (b) an endoscope (e.g., miniperc).

### Dual Lumen Exchange Catheter

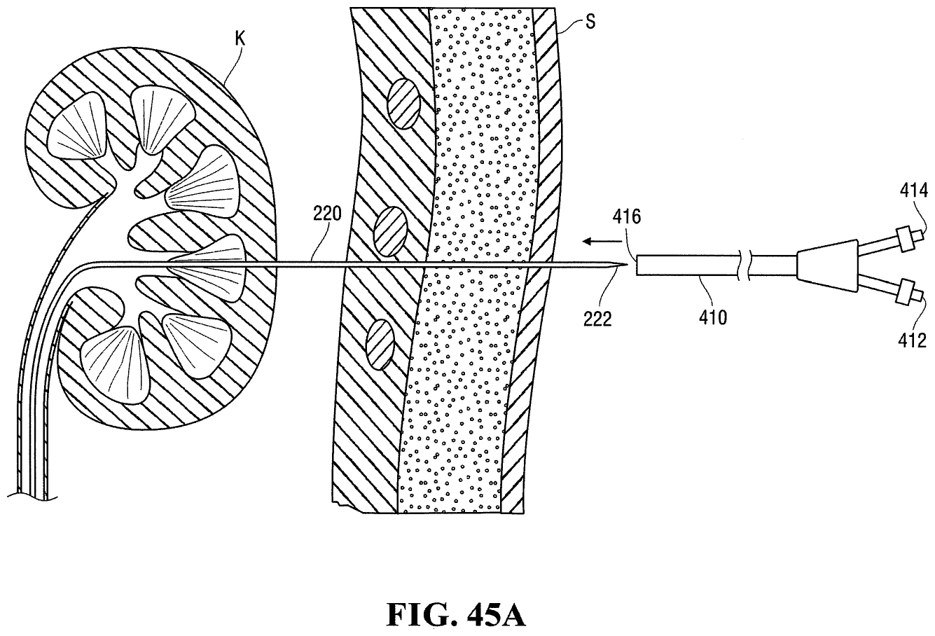

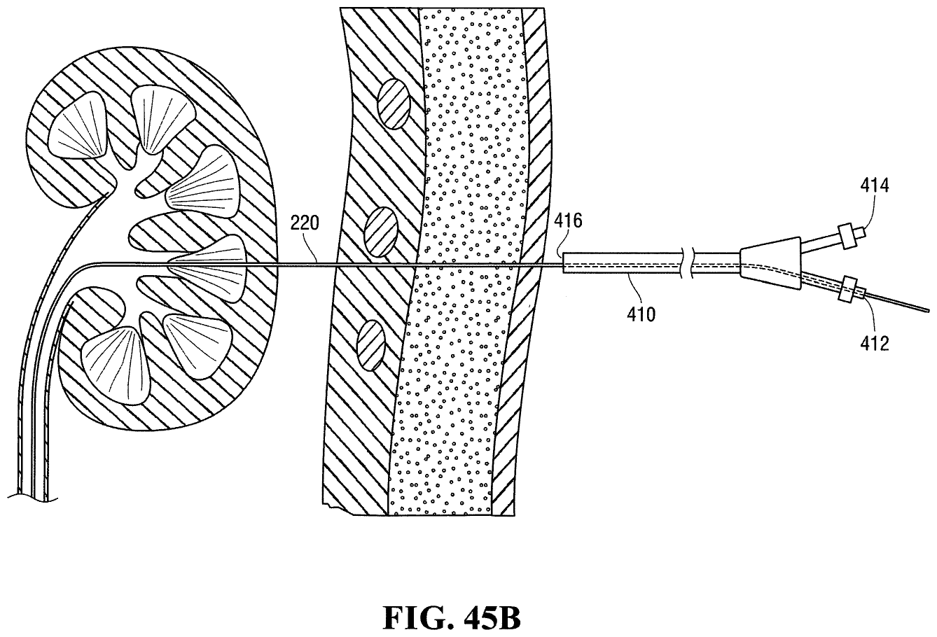

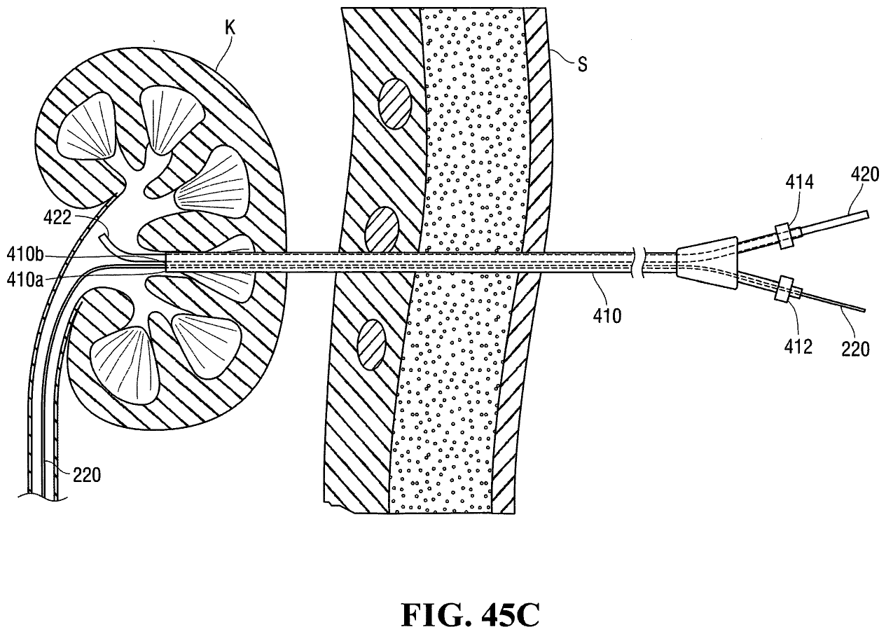

In the scenarios herein where an exchange catheter is discussed, a dual lumen exchange catheter is described. One embodiment of the exchange catheter is shown in FIG. 45A-45C. This exchange catheter 410 of FIG. 45A will allow advancement of the catheter 410 over the emergent nephrostomy puncture wire in a direction opposite the puncture wire emergence. One lumen of the dual lumen catheter, accessed via port 412, will be suitable for loading onto and advancement over the emergent nephrostomy wire 220 and the other lumen, accessed via port 414, in parallel to the first lumen, will permit passage of a new endourology working wire after the catheter tip 410a (and second lumen opening) is inside the renal collecting system.

The procedure for use of this exchange catheter is as follows:
1. Position a flexible ureteroscope in a selected calyx (see FIG. 28A).
2. Advance a nephrostomy puncture wire apparatus (puncture wire 220 with sheath) through a working channel of an ureteroscope until the tip emerges from the ureteroscope (see FIG. 28B).
3. Advance puncture wire until it is emergent outside of the flank (see FIG. 28C).
4. Load one lumen of the dual lumen exchange catheter 410 over the emergent puncture wire 220 (FIG. 45C) and control the puncture wire emergent out the back (proximal end) of the dual lumen catheter 410.
5. Advance the catheter 410 into the renal collecting system.
6. Advance a new endourology wire 420 (FIG. 45C) into the second lumen of the catheter 410 such that the tip 422 of the new wire 420 emerges from the end hole 410b of the second lumen and has entered the renal collecting system.

### Kits

The various components of the present invention, e.g., wires, sheaths, catheters, scopes, etc., can be packaged as various kits. Examples of such kits are as follows. Note other combinations of components, i.e., other kits, are also contemplated to facilitate performance of the methods disclosed above and illustrated in the drawings.

### Kit Example 1:

A nephrostomy puncture wire and tip protective sheath with releasable clamp as well as a separate dilator/sheath. A separate working endoscope may or may not be included.

### Kit Example 2:

A nephrostomy puncture wire and protective sheath with releasable clamp as well as an endoscope with a releasable inner dilator inside the endoscope lumen.

### Kit Example 3:

A nephrostomy puncture wire and protective sheath with releasable clamp, and a balloon nephrostomy tract dilation set and sheath. A separate endoscope may or may not be included.

### Kit Example 4:

A nephrostomy puncture wire and protective sheath with releasable clamp, along with an endoscope with a tapered tip.

### Kit Example 5:

A nephrostomy puncture wire and protective sheath with releasable clamp, along with a diagnostic and/or therapeutic catheter with a tapered tip.

### Kit Example 6:

A dual function puncture wire in a protective sheath with releasable clamp. The puncture wire could have multiple properties.

### Kit Example 7:

A nephrostomy wire and protective sheath with releasable clamp, and an endourology scope, e.g., for miniperc, microperc, endourologic surgery. This scope would have capabilities including connection to video, and one or more channels for irrigation/instrumentation. This scope may have a narrowed tip to permit atraumatic insertion into the kidney.

### Kit Example 8:

A nephrostomy puncture wire and protective sheath with releasable clamp, along with a separate wire clamp, e.g., tonsil clamp or Halstead style clamp, to control the emergent wire at the flank, and possibly to capture the wire while in a subcutaneous position. This clamp could be radio-opaque to aid in wire delivery under fluoroscopic guidance. This clamp could be compatible with the working channel of an endourology scope which may be included in the kit, such that the wire clamp could be inserted through the scope and used to capture the wire using the endoscope video and irrigation function.

### Kit Example 9:

A nephrostomy puncture wire and a wire exchange catheter whose tip is radiopaque either with a radiopaque tip alone (e.g., platinum band or similar) or with a radiopacifying agent mixed in the catheter material, in the form of either a single or coaxial system catheter system (to allow upsizing the puncture wire to a standard 0.035″ or 0.038″ endourology working wire).

### Kit Example 10:

Same as the kit example 9, with an endourology scope included.

### Kit Example 11:

A dual lumen exchange catheter. This kit may or may not include a nephrostomy puncture wire with protective sheath and releasable lock, and may include other items per above kits to effect the endourology procedure.

While the above description contains many specifics, those specifics should not be construed as limitations on the scope of the disclosure, but merely as exemplifications of preferred embodiments thereof. Those skilled in the art will envision many other possible variations that are within the scope and spirit of the disclosure as defined by the claims appended hereto.

Although the apparatus and methods of the subject disclosure have been described with respect to preferred embodiments, those skilled in the art will readily appreciate that changes and modifications may be made thereto without departing from the spirit and scope of the present disclosure as defined by the appended claims.

Additionally, persons skilled in the art will understand that the elements and features shown or described in connection with one embodiment may be combined with those of another embodiment without departing from the scope of the present invention and will appreciate further features and advantages of the presently disclosed subject matter based on the description provided.

Throughout the disclosure invention, terms such as “approximately,” “generally,” “substantially,” and the like should be understood to allow for variations in any numerical range or concept with which they are associated. For example, it is intended that the use of terms such as “approximately” and “generally” and “substantially” should be understood to encompass variations on the order of 25%, or to allow for manufacturing tolerances and/or deviations in design.

Although terms such as “first,” “second,” “third,” etc. may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present disclosure.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

What is claimed is:

1. A surgical method comprising the steps:
a) inserting a puncture wire and sheath in a first direction through a working channel in an ureteroscope, the puncture wire having a tissue penetrating tip shielded in a sheath;
b) advancing the puncture wire a first distance from the sheath and into a selected papilla and through a flank of a patient so the puncture wire has an emergent segment extending beyond the flank of the patient;
c) advancing a catheter over the emergent segment of the puncture wire outside the flank in a second direction opposite a direction of renal and flank puncture;
d) advancing the catheter into the flank in the second direction, the catheter having a lumen and a radiopaque portion to provide visibility of a tip of the catheter via fluoroscopic imaging;
e) removing the puncture wire from the catheter after the tip of the catheter is positioned in a desired region; and
f) advancing an endourology wire in the second direction through the catheter into the patient.

2. The method of claim 1, wherein the puncture wire is slidable within the sheath and releasably lockingly engageable therein, and the puncture wire is released from the sheath prior to the step of advancing the puncture wire through the flank.

3. The method of claim 1, wherein the endourology wire is advanced through the catheter and terminates in a kidney of the patient.

4. The method of claim 1, wherein the endourology wire is advanced through the catheter and terminates in a ureter of the patient.

5. The method of claim 4, wherein the puncture wire has a proximal segment with an outer diameter larger than a distal segment of the puncture wire to add stiffness to enable insertion of the catheter over the puncture wire and reduce or prevent kinking of the wire during advancement of the catheter.

6. The method of claim 5, wherein the proximal segment of the puncture wire is flexible to permit endoscope deflection of an endoscope positioned over the puncture wire.

7. The method of claim 1, wherein the radiopaque portion comprises a radiopaque band extending around at least a portion of a circumference of the catheter.

8. The method of claim 1, wherein the radiopaque portion comprises a radiopaque filler extending within a wall of the catheter along at least a portion of a length of the catheter.

9. A surgical method comprising the steps:
a) inserting a dual function puncture wire having a tissue penetrating tip shielded in a sheath in a first direction through a working channel in an ureteroscope;
b) advancing the puncture wire from the sheath a first distance from the sheath and through a flank of a patient so the puncture wire has an emergent segment extending beyond the flank of the patient;
c) advancing an outer member and dilator having a tapered tip over the emergent segment of the puncture wire outside the flank in a second direction opposite a direction of renal and flank puncture; and
d) removing the inner dilator leaving a distal end of the outer member in a kidney of the patient.

10. The method of claim 9, wherein the outer member comprises a nephroscope having a working channel for insertion of a surgical instrument for performing an endourologic procedure.

11. The method of claim 9, wherein the outer member comprises a sheath, and the method further comprises inserting an endoscope through the outer member, wherein the endoscope has a working channel for insertion of a surgical instrument for performing an endourologic procedure.

12. The method of claim 9, wherein the outer member comprises a balloon dilation catheter, and the method includes i) inflating a balloon of the balloon dilation catheter; ii) advancing a sheath over the balloon into the kidney; iii) deflating the balloon; iv) removing the balloon catheter leaving the sheath in place; and v) inserting an endoscope with a working channel through the outer sheath into the kidney.

13. The method of claim 11, wherein the dilator is removably positioned within the outer member.

14. The method of claim 11, wherein the dilator is integral with the outer member and extends distally.

15. The method of claim 14, wherein the outer member is an endoscope.

16. The method of claim 9, wherein the outer member comprises a catheter having multiple lumens and a mechanism to curl the catheter to secure the catheter in a kidney of the patient.

17. A surgical method comprising the steps:
a) inserting a puncture wire having a tissue penetrating tip shielded in a sheath in a first direction through a working channel in an ureteroscope;
b) advancing the puncture wire from the sheath a first distance from the sheath and through flank, fascia and fat of a patient so the puncture wire can emerge beyond a skin of the patient;
c) if misalignment of a distal segment of the puncture wire within the fat of the patient is detected, inserting a clamping member through the skin of the patient at a site spaced from an intended site of puncture wire emergence through the skin; and
d) grasping a distal region of the puncture wire with the clamping member and directing the puncture wire through the skin to complete a retrograde nephrostomy procedure.

18. The method of claim 17, wherein the clamping member is inserted directly through the skin of the patient.

19. The method of claim 17, further comprising the step of inserting a port through the skin of the patient at a site spaced from the intended site of puncture wire emergence and the step of inserting the clamping member comprises the step of inserting the clamping member through a lumen of the port, the port including illumination and visualization.

20. The method of claim 17, further comprising the step of after step d either a) releasing the puncture wire from the clamping member and positioning an exchange catheter over the puncture wire; or b) inserting an endoscope or catheter or sheath directly over the puncture wire into the kidney.
